(19) United States
(12) Patent Application Publication
(10) Pub. No.: US 2010/0330019 A1
(43) Pub. Date: Dec. 30, 2010

(54) KERATIN DERIVATIVES AND METHODS OF MAKING THE SAME

(76) Inventors: Robert James Kelly, Christchurch (NZ); Sonya Mary Scott, Christchurch (NZ); Alisa Dawn Roddick-Lanzilotta, Canterbury (NZ); Steven Geoffrey Aitken, North Canterbury (NZ)

Correspondence Address: VINSON & ELKINS, L.L.P., FIRST CITY TOWER, 1001 FANNIN STREET, SUITE 2500, HOUSTON, TX, 77002-6760, US

(21) Appl. No.: 12/740,633

(22) PCT Filed: Oct. 31, 2008
(86) PCT No.: PCT/US08/82025

§ 371 Date: Sep. 16, 2010

Related U.S. Application Data
(60) Provisional application No. 61/001,111, filed on Oct. 31, 2007.

(51) Int. Cl. A61K 8/65; C07K 14/78; A61Q 5/00; A61Q 19/00; A61Q 5/12; A61Q 5/02; A61Q 19/10; A61Q 19/08

(52) U.S. Cl. 424/70.14; 530/357; 514/18.8; 510/119; 510/130

(57) ABSTRACT

Soluble keratin derivatives are disclosed. The soluble keratin derivatives may include a soluble keratin protein having at least one substituted chemical group at a lysine group, terminal amine group and/or hydroxyl amino acid group of a soluble keratin protein. Soluble keratin derivatives may be formed by succinylation or quaternisation, or by reaction with fatty acid derivatives. The soluble keratin derivatives may be used in personal care formulations, and may also comprise mixtures of several different soluble keratin derivatives.

This application claims priority to U.S. Provisional Application No. 61/001,111, filed Oct. 31, 2007.

### FIELD OF THE INVENTION

The present invention is directed to soluble keratin derivatives formed by substitution of at least one chemical group at a lysine group, terminal amine group and/or hydroxyl amino acid group of a soluble keratin protein. The substituted chemical group may include an electrical charge. Soluble keratin derivatives may be formed by succinylation or quaternisation, or by reaction with fatty acid derivatives. The present invention is also directed to methods of preparation and use of the soluble keratin derivatives.

### BACKGROUND OF THE INVENTION

Keratin proteins are well known in the art and are found in a number of sources comprising wool, feathers and hair. Keratin fibers consist of a complex mix of related proteins that are all part of the keratin family. These proteins, often referred to as keratin protein fractions, can be grouped according to their structure and role within the fiber in to the following groups:
The intermediate filament proteins (IFP) which are fibrous proteins found mainly in the fiber cortex;
High sulfur proteins (HSP) which are globular proteins found in the matrix of the fiber cortex, as well as in the cuticle;
High glycine-tyrosine proteins (HGTP), found mainly in the fiber cortex.

The ultra structure of keratin fibers is well known in the art and is discussed in detail by R. C. Marshall et al, Structure and Biochemistry of Mammalian Hard Keratin, Electron Microscopy Reviews, (1991) 4, 47.

Keratin proteins are used in a wide variety of applications, including their use in personal care formulations, wound care applications, as orthopedic materials, and in the production of polymer films.

The keratin proteins perform a number of functions including conditioning, film forming, as humectants and as emollients.

The most commonly used keratin proteins are hydrolyzed in order to impart sufficient solubility to facilitate inclusion in a formulation. Keratin proteins are inherently insoluble due to the crosslinks associated with the characteristically high degree of cysteine present in the keratin protein. A problem in the art is that many of the desirable properties of the keratin proteins are lost upon hydrolysis, such as functionality. Numerous examples of the use of hydrolyzed proteins, including keratins, in personal care formulations are known in the art.

WO 98/51265 discloses the use of hydrolyzed proteins and their derivatives, particularly those with high sulfur content, in formulations to protect hair from the insults of environmental and chemical damage. The inventors in WO98/51265 use a combination of hydrolyzed proteins and a polyamino cationic agent in order to prepare the desired formulations.

U.S. Pat. No. 4,948,876 describes an S-sulphocysteine keratin peptide produced by enzymatic hydrolysis for use as an auxiliary in the dyeing of wool and hair. Enzymatic digestion is used by the authors to prepare low molecular weight peptides and achieve the desired solubility.

U.S. Pat. No. 4,895,722 describes the use of a range of keratin decomposition products, including those obtained by chemical and enzymatic hydrolysis, for the preparation of cosmetic products.

In the prior art described in which keratin proteins are used as a cosmetic ingredient, the keratin utilized is hydrolyzed as one material, with no attempt at fractionating the keratin source into its constituent components (e.g., IFP, HSP, HGTP). As a result of hydrolysis, many of the desirable properties of the keratin proteins are lost. Low molecular weight keratin peptides aggregate with a much lower degree of order to produce materials with much poorer physical properties than the high molecular weight keratins from which they are derived. In addition, irreversible conversion of cysteine as may occur with chemical methods of keratin decomposition yields a peptide product that has lost the core functionality that distinguishes it from other protein materials.

As taught in U.S. Published Patent Application No. 2006/0165635, incorporated herein by reference, intact keratins maintain many of the desirable characteristics of the native keratins from which they are derived and possess reactivity towards keratin substrates. Derivatives of these intact proteins are not taught in U.S. Published Patent Application No. 2006/0165635.

Chemicals such as quaternary ammonium compounds, succinylates and fatty acid derivatives are often used in personal care products to impart beneficial cosmetic properties, such as to condition hair or skin, to provide substantivity to skin or to bring surfactant character to a formulation. However, these chemical classes do not have benefits associated with proteins and peptides, and a problem exists to deliver both the benefit associated with the synthetic chemical and the benefit inherent in the proteinaceous material.

Chemical modification provides a useful method of modifying the functional properties of proteins. The chemical reactions commonly used to achieve this are acylation, succinylation, esterification, oxidation, reduction, glycosylation, phosphorylation and alkylation. These reactions usually involve the ionizable amino acid groups and the terminal amino groups.

Succinylation is commonly used in food proteins to improve solubility, foaming and emulsifying properties and also taste. The succinylation of a protein involves the introduction of negatively charged carbonyl groups which affect the electrostatic repulsive forces in the molecule, causing enhanced electrostatic repulsion between surfaces coated with protein resulting in greater emulsion stability. Succinylation reactions involve the amine groups in the protein and to a lesser degree, hydroxyl amino acids.

Another chemical modification is the step of quaternisation which results in the addition of a positively charged quaternary ammonium salt to the protein producing a more cationic species. Cationic surfactants are less effective detergents or foaming agents but they have two very important properties. Their positive charge allows them to absorb on to negatively charged substrates giving them antistatic behavior and softening action while some are also bactericides. They are often found in hair care products, such as conditioners.

A further chemical modification is to attach a fatty acid molecule to the amine groups on the protein molecule and therefore increase the hydrophobic character of the protein.

It would therefore be desirable to provide keratin derivatives that comprise cosmetic properties such as to condition hair or skin, to provide substantivity to skin or to bring surfactant character to a formulation, whilst also retaining other desirable keratin protein characteristics.

### SUMMARY OF THE INVENTION

In a first embodiment of the instant disclosure, it has been discovered by the inventors of the present application that soluble keratin proteins may be modified to form soluble keratin derivatives by substituting a chemical group at a lysine group, at a terminal amine group, and/or at hydroxyl amino acids groups on the soluble keratin protein.

In one aspect of the first embodiment, substitution may be completed by a succinylation reaction where an anhydride reacts with one or more lysine groups, terminal amine group and/or the hydroxyl amino acids groups in the soluble keratin protein. This has the effect of making the overall charge more negative

In another aspect of the first embodiment, substitution may be completed by a quaternisation reaction where the chemical group may be a positively charged quaternary ammonium salt added to one or more lysine groups, terminal amine groups and/or hydroxyl amino acids groups on the soluble keratin protein. This has the effect of making the overall charge more positive

In still another aspect of the first embodiment, substitution may occur by adding a long chain fatty acid to one or more lysine groups, terminal amine groups and/or hydroxyl amino acids groups on the soluble keratin protein, thereby neutralizing at least some of the protein charge. The long chain fatty acid may be a long chain fatty acid chloride, such as that formed by combining lauric acid and oxalyl chloride. Alternatively, the fatty acid derivative may be produced via a coupling process. A preferred coupling agent is ethylcarbodiimide hydrochloride (EDC) or N-(3-Dimethylaminopropyl)-N′-ethylcarbodiimide hydrochloride. In the above cases, the electrostatic repulsive forces in the molecule are altered resulting in enhanced surfactant and other properties.

The soluble keratin protein used in the first embodiment may be whole keratin or a keratin protein fraction. Examples of keratin protein fractions include the IFP fraction, the HSP fraction, and the HGTP fraction. The soluble keratin protein may be intact. The soluble keratin protein may instead be partly or fully hydrolyzed. The soluble keratin protein may be S-sulfonated keratin or partially oxidized keratin. In one aspect, the soluble keratin may be intact S-sulfonated keratin intermediate filament protein fraction. The cysteine content of the soluble keratin protein may be approximately 4%.

A second embodiment of the present disclosure is directed to a method for preparing a soluble keratin derivative by the step of substituting a chemical group at one or more lysine groups, terminal amine groups and/or hydroxyl amino acids groups of the soluble keratin protein. The method may comprise the steps of preparing an aqueous solution of soluble keratin protein and then mixing the aqueous solution with a solution containing the chemical group. The substituted chemical group may comprise a negatively charged group or alternatively a positively charged group which impart their charge to the soluble keratin protein. The soluble keratin protein may be similar to the soluble keratin protein described above in the first embodiment. Other optional components may be added to alter the end product properties, such as pH adjusters and pH buffer solutions. The method may also involve control of the reaction temperature.

In one aspect of the second embodiment, the substitution may comprise a succinylation reaction. Substitution in the succinylation reaction results in an anhydride reacting with one or more lysine groups, terminal amine group and/or hydroxyl amino acids groups of the soluble keratin protein to thereby form the soluble keratin derivative. The method may comprise the steps of preparing an aqueous solution of soluble keratin protein and then mixing the aqueous solution with a solution containing the anhydride.

In another aspect of the second embodiment, the substitution may comprise a quaternisation reaction. Substitution in the quaternisation reaction results in a positively charged quaternary ammonium salt added to one or more lysine groups, terminal amine group and/or hydroxyl amino acid groups in the soluble keratin protein. The method may comprise the steps of preparing an aqueous solution of soluble keratin protein and then mixing the aqueous solution with a solution containing the quaternary ammonium salt.

In still another aspect of the second embodiment, the substitution may comprise an acid chloride substitution reaction or a coupling reaction. Substitution in the acid chloride method or coupling reaction results in a fatty acid group being added to one or more lysine groups, terminal amine group and/or hydroxyl amino acid groups in the soluble keratin protein. The method comprises the steps of preparing an aqueous solution of soluble keratin protein and then mixing the aqueous solution with a solution containing the long chain fatty acid. The long chain fatty acid may be a mixture of lauroyl chloride and lauric acid via the acid chloride method or by use of N-(3-Dimethylaminopropyl)-N′-ethylcarbodiimide hydrochloride (EDC) coupling agent.

The third embodiment of the present disclosure is directed to a surfactant product comprising a soluble keratin derivative. The soluble keratin derivative may be as described above in the first embodiment.

The fourth embodiment of the present disclosure is directed to a personal care formulation comprising a soluble keratin derivative. The personal care formulation may comprise about 0.001% to 50% by weight of a soluble keratin derivative. The ratio may be 0.001% to 10% or 0.001% to 5%. The soluble keratin derivative may be as described above in the first embodiment. Personal care formulations in which the soluble keratin derivative may be used on account of the soluble keratin derivative properties comprise any of the following: conditioning shampoo, body/facial cleanser/shampoo, hair conditioner, hair gel, hair mouse, hair setting lotion, hairspray, pre-perming solution, post-perming solution, moisturizing cream, shower gel, foaming bath gel, mascara, nail polish, liquid foundation, shaving cream, and lipstick. Other personal care formulations are also included within the invention (e.g., a detergent that protects skin).

The fifth embodiment of the present disclosure is directed to an additive for a personal care formulation. The additive may comprise the soluble keratin derivative as described above in the first embodiment.

The sixth embodiment of the present disclosure is a method for treating hair. The method may comprises the step of applying a personal care formulation comprising from about 0.001% to 50% of a soluble keratin derivative to hair. The soluble keratin derivative may be as described above in the first embodiment.

The seventh embodiment of the present disclosure is a method for treating hair. The method may comprises the step of applying a personal care composition comprising an additive to hair. The additive may comprise soluble keratin derivative. The soluble keratin derivative may be as described above in the first embodiment.

The eighth embodiment of the present disclosure is a soluble keratin derivative mixture. The soluble keratin derivative mixture may comprise two or more soluble keratin derivatives. The soluble keratin derivative mixture may comprise a first soluble keratin protein fraction with at least one substituted chemical group at a lysine group, at a terminal amine group, and/or at hydroxyl amino acids groups on the soluble keratin protein fraction. The soluble keratin derivative mixture may further comprise a second soluble keratin protein fraction with at least one substituted chemical group at a lysine group, at a terminal amine group, and/or at hydroxyl amino acids groups on the soluble keratin protein fraction. The first and second soluble keratin fractions may be intermediate filament protein, high sulfur protein or high glycine-tyrosine protein. The first soluble keratin protein fraction may be different from the second soluble keratin protein fraction.

The ninth embodiment of the present disclosure is a method of producing a soluble keratin derivative mixture. The method may comprise the step of mixing a first soluble keratin protein fraction with at least one substituted chemical group at a lysine group, a terminal amine group and/or a hydroxyl amino acid group on the first soluble keratin protein fraction with a second soluble keratin protein fraction with at least one substituted chemical group at a lysine group, a terminal amine group and/or a hydroxyl amino acid group on the second soluble keratin protein fraction. The first and second soluble keratin fractions may be intermediate filament protein, high sulfur protein or high glycine-tyrosine protein. The first soluble keratin protein fraction may be different from the second soluble keratin protein fraction.

### BRIEF DESCRIPTION OF THE DRAWINGS

Further aspects of the present invention will become apparent from the following description which is given by way of example only and with reference to the accompanying drawings in which:

FIG. 1 shows a graph indicating the charge characteristics of succinylated protein samples where 0%=non-derivatized protein (intact keratin), 28%=sample SPA, 74%=sample SPB, 79%=sample SPC and 83%=sample SPD;

FIG. 2 shows a pH-solubility curve for intact keratin and succinylated proteins;

FIG. 3 shows a graph indicating the charge characteristics of quaternised protein samples where 0%=non-derivatized protein (intact keratin), 7%=sample QuatA, 41%=sample QuatB, 65%=sample QuatC and 85%=sample QuatD;

FIG. 4 shows a pH-solubility curve for intact keratin and quaternised proteins;

FIG. 5 shows scanning electron microscope (SEM) images of untreated hairs (Samples E and F) (Mag: 800×);

FIG. 6 shows SEM images of untreated hairs (Samples E and F) (Mag: 2000×);

FIG. 7 shows SEM images of sodium laureth sulfate (SLES) washed hairs (Samples A and B) (Mag: 800×);

FIG. 8 shows SEM images of SLES Washed Hairs (Samples A and B) (Mag: 2000×);

FIG. 9 shows SEM images of succinylated keratin protein sample SPC washed hairs (Samples C and D) (Mag: 800×);

FIG. 10 shows SEM images of SPC washed hairs (Samples C and D) (Mag: 2000×);

FIG. 11 shows TLC analysis of the extracted hair lipids for the different hair samples (A-F) where CE, cholesterol ester; FFAE, fatty acid ester; FFA, free fatty acid; Chol, cholesterol; Cer, ceramide; TG, triglycerides;

FIG. 12 shows the average combing stroke force calculation [in this example calculated as=(100+160+170+180+200)/5=162] and the graph used in calculating average combing force for each force/elongation curve;

FIG. 13 shows a graph of the mean values of the combing force measurement for treated and untreated hair tresses on the two experiments;

FIG. 14 shows a graph of the mean values of the highest peak measured for the combing force found for the treated and untreated hair tresses on the two experiments

FIG. 15 shows a graph of the mean values of the highest peak reported on the combing force measurement for treated and untreated hair tresses on the two experiments;

FIG. 16 shows a selection percentage of the different questions of all judges for the different hair tresses (untreated and treated) for high molecular weight quaternised zo derivative; and,

FIG. 17 shows a selection percentage of the different questions of all judges for the different hair tresses (untreated and treated) for low molecular weight quaternised derivative.

### DETAILED DESCRIPTION OF THE PREFERRED EMBODIMENTS

In a first embodiment of the present disclosure, a soluble keratin derivative is disclosed. The soluble keratin derivative comprises a modification to a soluble keratin protein whereby the soluble keratin protein has been modified to form derivatives by substituting a chemical group at one or more lysine groups, terminal amine groups and/or hydroxyl amino acids groups on the soluble keratin protein.

Keratin is a family of proteins characterized by a high amount of the amino acid cystine, which imparts a high degree of crosslinking to keratin proteins through disulfide links. Keratin proteins are also highly ordered proteins providing a fundamental structural role to many biological tissues.

Furthermore, the occurrence of disulfide crosslinks provides a degree of resilience to enzymatic degradation within the body, allowing any material delivered in the keratin to be maintained at a particular site for a controllable period of time.

Because keratin is naturally insoluble, keratin must be chemically modified to produce soluble keratin protein. Any keratin modified to be soluble may be used in the present invention, just as any method for solubilising keratin known in the art may be used to provide a soluble keratin for use in the present invention.

One such process involves chemically modifying keratin to form S-sulfonated keratin as described in U.S. Pat. No. 7,148,327, incorporated herein by reference.

In one aspect of the first embodiment, the soluble keratin is S-sulfonated keratin protein. S-sulfonated keratin refers to keratin protein that undergoes a process wherein the disulfide bonds between cystine amino acid in keratin protein are reversibly modified to create polar functional groups that allow for controlled re-introduction of the natural disulfide crosslinks originally present in the keratin protein. S-sulfonated keratins have cysteine/cystine present predominantly in the form of S-sulfocysteine. This highly polar group imparts a degree of solubility to proteins. Whilst being stable in solution, the S-sulfo group is a labile cysteine derivative, highly reactive towards thiols, such as cysteine, and other reducing agents. Reaction with reducing agents leads to conversion of the S-sulfocysteine group back to cystine. S-sulfocysteine is chemically different from cysteic acid, although both groups contain the SO₃⁻ group. Cysteic acid is produced irreversibly by the oxidation of cysteine or cystine and once formed cannot form disulfide crosslinks back to cysteine. S-sulfocysteine is reactive towards cysteine and readily forms disulfide crosslinks.

In another aspect of the first embodiment, the soluble keratin is partially oxidized keratin protein. Partially oxidized means that >85% of the cystines in the keratin have been oxidised to cysteic acids, in addition to possibly a relatively small number of other oxidation sensitive amino acids. Partial oxidation of keratin protein results in solubilising the keratin protein by the conversion of the disulfide bonds between cystine amino acid in keratin protein to cysteic acid.

The soluble keratin protein of the first embodiment may be whole keratin protein that has not been separated into differing fractions. In an alternative embodiment, the keratin protein may be a keratin protein fraction. The hard alpha keratin proteins such as those derived from human hair, wool, animal fibers, horns, hooves or other mammalian sources, can be classified into particular components according to their biochemical properties, specifically their molecular weight and amino acid composition. U.S. Published Patent Application No. 2006/0165635 describes the particular compositions in detail and is incorporated herein by reference. Keratin protein fractions identified above may be classified into distinct groups from within the keratin protein family, and comprise: intermediate filament proteins (IFP), high sulfur proteins (HSP) and high glycine-tyrosine proteins (HGTP).

Intermediate filament proteins are described in detail by Orwin et al. (Structure and Biochemistry of Mammalian Hard Keratin, Electron Microscopy Reviews, 4, 47, 1991) and also referred to as low sulfur proteins by Gillespie (Biochemistry and physiology of the skin, vol. 1, Ed. Goldsmith Oxford University Press, London, 1983, pp. 475-510). Key characteristics of the intermediate filament protein family are molecular weight in the range 40-60 kD and a cysteine content (measured as half cystine) of around 4%.

The high sulfur protein family is also well described by Orwin and Gillespie in the same publications referenced above. This protein family has a large degree of heterogeneity, but can be characterized as having a molecular weight in the range 10-30 kD and a cysteine content of greater than 10%. A subset of this family is the ultrahigh sulfur proteins, which can have a cysteine content of up to 34%.

The high glycine-tyrosine protein family is also well described by Orwin and Gillespie in the same publications referenced above. This family is also referred to as the high tyrosine proteins and has characteristics of a molecular weight less than 10 kD, a tyrosine content typically greater than 10% and a glycine content typically greater than 20%.

For the purpose of this invention, a ‘keratin protein fraction’ is a purified form of keratin that contains predominantly, although not entirely, one distinct protein group as described above.

The soluble keratin protein of the first embodiment may be intact. The term ‘intact’ refers to proteins that have not been significantly hydrolyzed, with hydrolysis being defined as the cleavage of bonds through the addition of water. Gillespie considers intact to refer to proteins in the keratinized polymeric state and further refers to polypeptide subunits which complex to form intact keratin in wool and hair. For the purposes of this specification, ‘intact’ refers to the polypeptide subunits described in Gillespie. These are equivalent to the keratin proteins in their native form without the disulfide crosslinks formed through the process of keratinization.

Intact keratin proteins and keratin protein fractions are discussed in greater detail in co-pending U.S. Patent Published Application No. 2008/0038327 and of which the entire application is hereby incorporated by reference.

The soluble keratin protein may be hydrolyzed. Hydrolysis refers to the cleavage of bonds through the addition of water. Keratin proteins hydrolyzed in this way may also be referred to as keratin peptides or oligo-peptides. For the purposes of this specification, the term hydrolyzed protein encompasses peptides. It should be appreciated that derivatization taught in this disclosure incorporates derivatizing both whole proteins and hydrolyzed proteins (peptides). By way of example, a reaction scheme understood by the inventors to occur in hydrolyzing is as shown in Scheme 1 below:

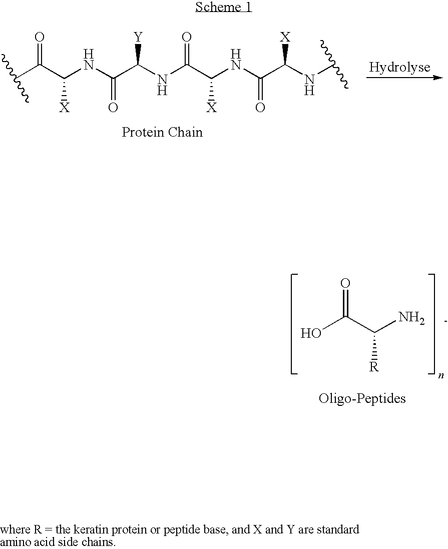

Scheme 1 illustrates hydrolyzation before derivatization although it should be appreciated that hydrolyzing may occur post derivatization instead and the above Scheme should not be seen as limiting.

It should also be appreciated that, unless noted or suggested otherwise (e.g., when referencing intact proteins), the term “protein” as used herein encompasses both whole proteins and peptides.

The soluble keratin protein may be in a solution, the solution being any suitable solution for use in a personal care formulation, such as water. The aqueous solution may be any ratio of soluble keratin to solution suitable for preparing an aqueous solution. The aqueous solution of soluble keratin protein may be from 0.001 to 50% by weight soluble keratin protein for a personal care formulation.

The chemical group used to produce the soluble keratin derivative may comprise a negatively charged group or alternatively a positively charged group which imparts its charge to the soluble keratin protein.

The chemical group may join to the soluble keratin protein at the location of one or more lysine groups, terminal amine groups, and/or hydroxyl amino acids groups of the soluble keratin protein. The chemical group attaches to the keratin by means of substituting with one or more lysine groups, terminal amine groups and/or hydroxyl amino acids groups of the soluble keratin protein.

In one aspect of the first embodiment disclosed herein, a soluble keratin derivative is disclosed wherein the soluble keratin protein has been modified via a succinylation reaction and may be referred to a soluble keratin succinylation derivative.

Substitution in the succinylation reaction results in an anhydride reacting with one or more of the lysine groups and/or the terminal amine groups in the protein and, to a lesser degree, the hydroxyl amino acids groups to form the soluble keratin derivative. In one embodiment, the substituted chemical group comprises:

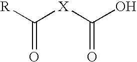

where R=the soluble keratin protein and X=an optionally substituted alkyl group. More specifically, X may be (CH₂)ₙ, where n may range from 2 to 6.

In a specific example, reactions utilizing a preferred reagent, succinic anhydride (X═CH₂CH₂), are understood to occur based on the following process as shown in Scheme 2 below:

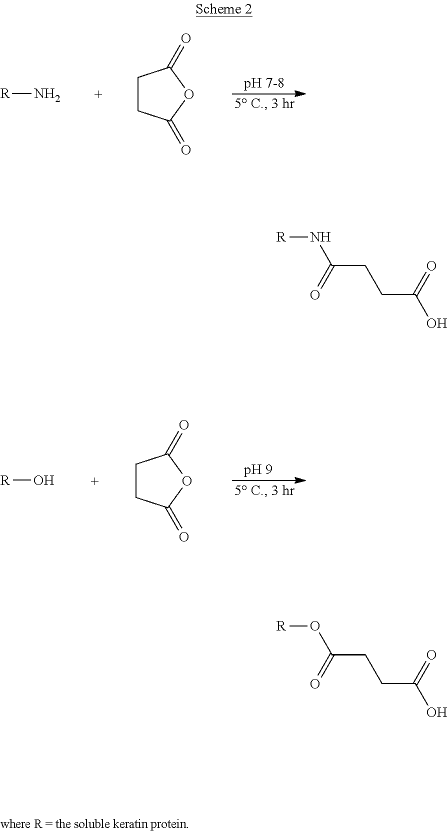

Succinylation may be completed using S-sulfonated intermediate filament keratin protein fraction and succinic anhydride. The succinic anhydride reacts with the primary amine groups in the S-sulfonated keratin protein fraction (lysine and N-terminals). The reaction may also occur to a lesser degree at the hydroxyl amino acids groups (serine, threonine and tyrosine). The various reactions give carboxylic acid functionalities. As should be appreciated, in the case of the lysine groups the reaction changes the soluble keratin protein from having an amino acid which is positive some of the time to having a negatively charged carboxylate group. This has the effect of making the soluble keratin protein more negatively charged.

The succinylation process may also be modified by using other reagents comprising, for example, other different anhydride compounds (e.g. phthalic, glutaric, butyric or acetic anhydride). Alternatively, p-toluenesulfonyl chloride may be used as the reagent to give a sulfamidated protein with aromatic rings attached.

In one aspect, succinic anhydride or other reagents may be added to the soluble keratin protein at a ratio from approximately 1 to 10 parts succinic anhydride to 100 parts soluble keratin protein. In a more specific example, succinic anhydride is added at a ratio of approximately 1 part succinic anhydride to 25 parts soluble keratin protein.

During the reaction step, the pH may be controlled to between 7.0 and 9.0. As the pH tends to reduce during the reaction, pH may be controlled by addition of pH increasing agent such as sodium hydroxide.

Also, during the reaction step, the temperature may be controlled to between approximately 1° C. and 10° C., more preferably, to around 5° C.

In another aspect of the first embodiment, a soluble keratin protein may be modified via a quaternisation reaction. Substitution in the quaternisation reaction results in a positively charged quaternary ammonium salt reacting with one or more lysine groups and/or terminal amine groups in the protein. The reaction may also occur to a lesser degree at the hydroxyl amino acids groups (serine, threonine and tyrosine). In one embodiment, the substituted chemical group comprises:

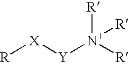

where R=the soluble keratin protein, X═NH or O, Y=an optionally substituted alkyl chain and R′=an alkyl chain. In a specific example, X may be NH, Y may be CH₂CH(OH)CH₂ and R′ may be CH₃.

In one specific example, reactions using a preferred reagent are understood to occur based on the following process as shown in Scheme 3 below:

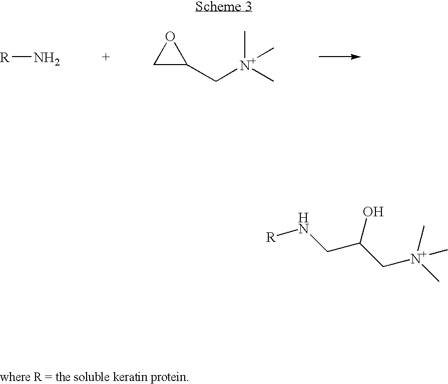

Quaternisation may be completed using glycidyl trimethyl ammonium chloride (GTMAC). The GTMAC reacts with the primary amine groups in the soluble keratin protein (lysine) and terminal amine groups in the soluble keratin protein (N-terminals). The reaction may also occur to a lesser degree at the hydroxyl amino acids groups (serine, threonine and tyrosine). As should be appreciated, in the case of the lysine groups the reaction changes the soluble keratin protein from having an amino acid which is positive some of the time to having a positively charged quaternary ammonium salt added to the lysine groups and the terminal amine groups in the soluble keratin protein. This has the effect of making the soluble keratin protein more positively charged.

Whilst GTMAC is described above, it should be appreciated that other quaternary salts may be used without departing from the scope of the invention, the key aim being that a reactive group is attached to the quaternary salt able to react with the soluble keratin protein. For example, other quaternary salts may be used, particularly those with an epoxide group attached comprising long chain salts such as C₁₀, C₁₂, C₁₄, C₁₆, C₄₀ and longer. As noted, an epoxide group is favorable. This is because this group is highly reactive and the long chain of the protein attaches to the quaternary nitrogen, most usually giving molecules of the form R₁—N(CH₂)ₙR₂ where R₁ is keratin protein or peptide, and R₂ is the quaternary nitrogen containing moiety.

In one aspect, GTMAC may be added to the soluble keratin protein at a ratio from approximately 1 to 10 parts GTMAC to 80 parts soluble keratin protein. In one specific example, GTMAC is added at a ratio of approximately 1 part GTMAC to 16 parts soluble keratin protein.

During the reaction step, the temperature may be controlled at approximately 40° C.

In one embodiment, GTMAC may be added to hydrolyzed soluble keratin proteins at a ratio suitable to result in greater than 85% substitution of all terminal and lysine side chain amines as determined by OPA analysis.

In still another aspect of the first embodiment, a soluble keratin derivative with a long chain fatty acid is disclosed. Substitution in this aspect results in negatively charged fatty acid groups being added to one or more lysine groups and/or terminal amine groups of the protein. The reaction may also occur to a lesser degree at the hydroxyl amino acids groups (serine, threonine and tyrosine). The term ‘long chain’ refers to the fatty acid being a C₁₀ or greater length. Preferably, the fatty acid is a C₁₀₋₁₈ chain. In one aspect, the substituted chemical group comprises:

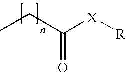

where R=the soluble keratin protein, X═NH or O,

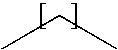

=a repeating fatty acid chain and n=10 to 40. In a specific example, X may be NH,

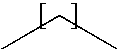

may be within the range of 10 to 18.

In a specific example, reactions using a preferred reagent are understood to occur based on the following process as shown in Scheme 4 below:

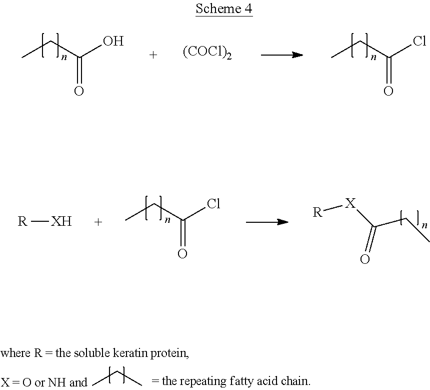

In the above process, the long chain fatty acid is a fatty acid chloride such as that formed by combining lauric acid and oxalyl chloride. In further embodiments, other reagents instead of oxalyl chloride may be used (e.g., thionyl chloride, inorganic halides and reagents generally with the group COCl). In this alternative, the reaction is kept at a temperature of between 1° C. and 10° C. for the duration of the protein reaction and the pH is maintained at around 8.

Alternatively, the fatty acid derivative may be produced via a coupling process. Coupling reactions using a preferred reagent are understood to occur based on the following process as shown in Scheme 5 below:

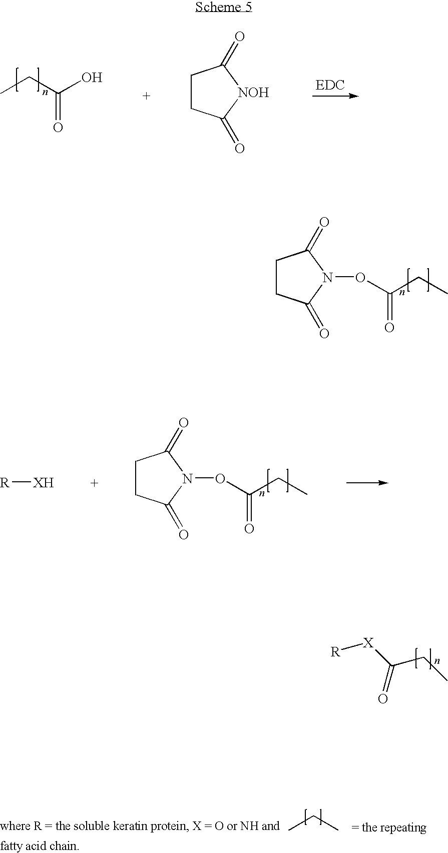

In the above process, the preferred coupling agent is EDC or N-(3-Dimethylaminopropyl)-N′-ethylcarbodiimide hydrochloride. Other coupling agents known in the art may also be used without departing from the scope of the invention.

In one aspect, fatty acids are added to hydrolyzed keratin proteins at a ratio suitable to result in greater than 85% substitution of all terminal and lysine side chain amines as determined by OPA analysis.

In a second embodiment of the present disclosure, a method for preparing a soluble keratin derivative is disclosed. The method comprises the step of substituting a chemical group to one or more lysine groups, terminal amine groups and/or hydroxyl amino acids groups of the soluble keratin protein. More specifically, the method comprises the steps of preparing an aqueous solution of soluble keratin protein and then mixing the aqueous solution with a solution containing the chemical group. The chemical group may comprise a negatively charged group or alternatively a positively charged group which imparts its charge to the soluble keratin protein. Other optional components may be added to alter the end product properties, such as pH adjusters and pH buffer solutions. The method also may involve control of the reaction temperature.

In one aspect of the second embodiment, the method for preparing a soluble keratin derivative comprises a step of completing a succinylation reaction. Substitution in the succinylation reaction results in an anhydride reacting with one or more lysine groups and/or terminal amine groups in the soluble keratin protein and to a lesser degree, the hydroxyl amino acids groups to form the soluble keratin derivative. The method comprises the steps of preparing an aqueous solution of soluble keratin protein and then mixing the aqueous solution with a solution containing the anhydride.

Succinylation may be completed using succinic anhydride. The succinic anhydride reacts with the primary amine groups in the soluble keratin protein (lysine and N-terminals) and to a lesser degree, hydroxyl amino acids (serine, threonine and tyrosine) to give carboxylic acid functionalities. Other reagents as discussed previously may also be used.

Succinic anhydride may be added to the soluble keratin protein at a ratio from approximately 1 to 10 parts succinic anhydride to 100 parts soluble keratin protein. In one specific example, succinic anhydride is added at a ratio of approximately 1 part succinic anhydride to 25 parts soluble keratin protein.

During the reaction step, the pH may be controlled to between 8.0 and 8.2. As the pH tends to reduce during the reaction, pH may be controlled by addition of a pH increasing agent, such as sodium hydroxide.

Also, during the reaction step, the temperature may be controlled to between approximately 1° C. and 10° C., more preferably, to around 5° C.

In another aspect of the second embodiment of the present disclosure, the method for preparing a soluble keratin derivative comprises the step of a quaternisation reaction. Substitution in the quaternisation reaction results in a positively charged quaternary ammonium salt reacting with the lysine groups and the terminal amine groups in the soluble keratin protein. The method comprises the steps of preparing an aqueous solution of soluble keratin protein and then mixing the aqueous solution with a solution containing the quaternary ammonium salt.

Quaternisation may be completed using glycidyl trimethyl ammonium chloride (GTMAC). The GTMAC reacts with the primary amine groups in the soluble keratin protein (lysine) and terminal amine groups in the soluble keratin protein (N-terminals). The reaction may also occur to a lesser degree at the hydroxyl amino acids groups (serine, threonine and tyrosine). Other quaternary salts as discussed previously may also be used.

GTMAC may be added to the soluble keratin protein at a ratio from approximately 1 to 10 parts GTMAC to 80 parts soluble keratin protein. In one example, GTMAC may be added at a ratio of approximately 1 part GTMAC to 16 parts soluble keratin protein.

During the reaction step, the temperature may be controlled at approximately 40° C.

In still another aspect of the second embodiment, the method of preparing a soluble keratin derivative may comprise the step of an acid chloride method or an EDC coupling reaction. Substitution in the acid chloride method or EDC coupling reaction results in a fatty acid group being added to one or more lysine groups and/or terminal amine groups in the soluble keratin protein. The reaction may also occur to a lesser degree at the hydroxyl amino acids groups (serine, threonine and tyrosine). The method comprises the steps of preparing an aqueous solution of soluble keratin protein and then mixing the aqueous solution with a solution containing the long chain fatty acid. The long chain fatty acid may be lauroyl chloride produced via an acid chloride method or lauric acid which is used in conjunction with the coupling agent N-(3-Dimethylaminopropyl)-N′-ethylcarbodiimide hydrochloride (EDC).

During the preferred acid chloride method, the temperature of the reaction solution may be maintained at between approximately 1° C. and 10° C. and kept at a pH of approximately 8.

In a third embodiment, a surfactant product comprising soluble keratin derivative is disclosed. The soluble keratin derivatives disclosed herein have surfactant type properties comprising the ability to reduce the surface tension of a liquid such as water, thereby allowing easier spreading and reducing interfacial tension between different phases. This is understood to be because the soluble keratin derivatizes of the present invention are amphiphilic, having both hydrophobic ‘tails’ and hydrophilic ‘heads’. This means that they are soluble in both organic solvents and water. Whilst base keratin proteins also exhibit some degree of surfactant properties, the soluble keratin derivatives of the present disclosure exhibit much stronger surfactant properties due to the altered charge caused by the substitution reactions. For example, half the concentration of soluble keratin derivative according to the instant disclosure may be used to achieve the same degree of reduction in water surface tension as compared to base keratin protein. In addition, foaming (another property of surfactants) is much greater and longer lasting with the soluble keratin derivatives of the instant disclosure than with the base keratin protein, even with markedly decreased concentrations of soluble keratin derivative compared to the base keratin protein. Soluble keratin derivative may be used alone as a surfactant in a formulation. In an alternative, soluble keratin derivative is used in conjunction with other surfactants in formulations.

In a fourth embodiment of the instant disclosure, a personal care formulation comprising a soluble keratin derivative is disclosed. The term ‘personal care formulation’ includes any substance or preparation intended for placement in contact with any external part of the human body, including the mucous membranes of the oral cavity and the teeth, with a view to achieving an effect comprising: altering the odors of the body, changing the appearance of the body, cleansing the body, maintaining the body in good condition, or perfuming the body.

The personal care formation may contain about 0.001% to 50% by weight of a soluble keratin derivative. The ratio is preferably 0.001% to 10% by weight and more preferably 0.001% to 5% by weight. The personal care formulation may further comprise any suitable cosmetic carrier.

The soluble keratin derivative may be the soluble keratin derivative as described in detail above in the first embodiment.

Personal care formulations in which the keratin derivative may be used on account of the soluble keratin derivative properties comprise any of the following: conditioning shampoo, body/facial cleanser/shampoo, hair conditioner, hair gel, hair mouse, hair setting lotion, hairspray, pre-perming solution, post-perming solution, moisturizing cream, shower gel, foaming bath gel, mascara, nail polish, liquid foundation, shaving cream, and lipstick. Other personal care formulations that assist in achieving the properties noted above are also encompassed within the invention for example a detergent that protects skin from drying.

In a fifth embodiment of the instant disclosure, an additive for a personal care formulation comprising a soluble keratin derivative is disclosed. The soluble keratin derivative may be the soluble keratin derivative as described in detail above in the first embodiment. The additive may be added to any suitable personal care formulation, such as those described above in the fourth embodiment. The additive may be added to the personal care formulation in an amount ranging from 0.1 to 5% by weight of the personal care formulation. The personal care formulation may also comprise any suitable cosmetic carrier.

In a sixth embodiment, a method of treating hair is disclosed. The method may comprise the step of applying a personal care formulation comprising from about 0.001% to 50% of a soluble keratin derivative to hair. The soluble keratin derivative may be the soluble keratin derivative described above in the first embodiment. Any suitable personal care formulation may be used, such as any of those described above. The personal care formulation used in the method of the sixth embodiment may be applied to any type of hair in any suitable quantity.

In a seventh embodiment, an alternate method of treating hair is disclosed. The method may comprise the step of applying a personal care formulation comprising an additive to hair. The additive may comprise a keratin protein derivative. The keratin protein derivative may be the keratin protein derivative described above in the first embodiment. Any suitable amount of additive may be included in the personal care formulation and any suitable amount of personal care formulation may be applied to hair. The additive-containing personal care formulation may be applied to any type of hair and may be any of the personal care formulations described above.

In an eighth embodiment, a soluble keratin derivative mixture is disclosed. The soluble keratin mixture may comprise two or more soluble keratin derivatives mixed together. Mixtures of soluble keratin derivatives may have a favorable volume and cysteine content. Increased cysteine content (specifically S-sulfonated Cys and oxidized Cys (Cysteic acid)) may result in improved efficacy of the materials as personal care formulations. Improved volume may result in the manufacturing process being more commercially viable.

The soluble keratin derivatives may be any of those described above in the first embodiment. In one aspect of this embodiment, the soluble keratin derivatives are soluble keratin protein fractions having substituted chemical groups as described in greater detail above. The soluble keratin protein fraction of the soluble keratin derivatives used in the mixture may be intermediate filament protein, high sulfur protein or high glycine-tyrosine protein. The soluble keratin protein fractions may be S-sulfonated or partially oxidized. The soluble keratin protein fraction may also be intact or hydrolysed as discussed in greater detail above.

The mixture of soluble keratin derivatives may comprise soluble keratin derivatives having different keratin protein fractions. In other words, if the soluble keratin derivative mixture comprises a first soluble keratin derivative comprising keratin protein fraction with substituted chemical groups and a second soluble keratin derivative comprising keratin protein fraction with substituted chemical groups, the keratin protein fraction of the first soluble keratin derivative may be different from the keratin protein fraction of the second soluble keratin derivative. In one specific example, the keratin protein fraction of the first soluble keratin derivative may be keratin intermediate filament protein while the keratin protein fraction of the second soluble keratin derivative may be either keratin high sulfur protein or keratin high glycine-tyrosine protein. Any combination of the keratin protein fractions may be used.

In another aspect of this embodiment, the ratio of different soluble keratin derivatives within the soluble keratin derivative mixture may be selected according to the soluble keratin fraction component of each of the soluble keratin derivatives. Where the first soluble keratin derivative comprises intermediate filament protein and the second soluble keratin derivative comprises either high sulfur protein or high glycine-tyrosine protein, the ratio of first soluble keratin derivative to second soluble keratin derivative may be any suitable ratio. In one aspect, the ratio is determined by the keratin source used.

In a ninth embodiment, a method of producing a soluble keratin derivative mixture is disclosed. The method may generally comprise mixing two or more soluble keratin derivatives together. In one aspect of this embodiment, the soluble keratin derivatives are soluble keratin protein fractions having substituted chemical groups as described in greater detail above. The soluble keratin protein fraction of the soluble keratin derivatives used in the mixture may be intermediate filament protein, high sulfur protein or high glycine-tyrosine protein. The soluble keratin protein fractions may be S-sulfonated or partially oxidized. The soluble keratin protein fraction may also be intact or hydrolysed as discussed in greater detail above.

The soluble keratin derivatives mixed together in the method of the ninth embodiment may comprise soluble keratin derivatives having different keratin protein fractions. In other words, if the soluble keratin derivative mixture comprises a first soluble keratin derivative comprising keratin protein fraction with substituted chemical groups mixed with a second soluble keratin derivative comprising keratin protein fraction with substituted chemical groups, the keratin protein fraction of the first soluble keratin derivative may be different from the keratin protein fraction of the second soluble keratin derivative. In one specific example, the keratin protein fraction of the first soluble keratin derivative may be keratin intermediate filament protein while the keratin protein fraction of the second soluble keratin derivative may be either keratin high sulfur protein or keratin high glycine-tyrosine protein. Any combination of the keratin protein fractions may be used in the method of making the soluble keratin derivative mixture.

In another aspect of this embodiment, the different soluble keratin derivatives may be mixed together at certain ratios based on the soluble keratin fraction component of each of the soluble keratin derivatives. For example, if a first soluble keratin derivative comprising intermediate filament protein is mixed with a second soluble keratin derivative comprising either high sulfur protein or high glycine-tyrosine protein, the ratio of first soluble keratin derivative to second soluble keratin derivative may be may be any suitable ratio. In one aspect, the ratio is determined by the keratin source used.

### WORKING EXAMPLES

### Example 1

### Manufacturing a Succinylated Keratin Derivative

This Example describes investigations into the derivatization of soluble keratin proteins. It describes the procedures by which the soluble keratin proteins are succinylated and the resulting derivative properties.

Succinylation of intact soluble keratin intermediate filament protein was performed by the addition of succinic anhydride to the reaction. Succinic anhydride reacts with the primary amine groups in the intact soluble keratin IFP (lysine and N-terminals) and to a lesser degree, hydroxyl amino acids groups (serine, threonine and tyrosine) to give carboxylic acid functionalities. As should be appreciated, in the case of the lysine groups it means an amino acid which is positive some of the time has been substituted with a negatively charged carboxylate group. This should have the effect of making the intact soluble keratin IFP even more negative in character.

More specifically, the method was completed by the steps of:
(i) 100 g of intact soluble keratin IFP (3.2% solution) at pH of 8 was cooled to 5° C. in a water bath;
(ii) 8.3 g of succinic anhydride was added over the period of 1 hour. The pH was maintained between 8 and 8.2 by the continuous addition of 1 molL⁻¹ NaOH during the reaction;
(iii) Once the pH had stopped changing, the solution was stirred for 1 hour;
(iv) acid was added to reduce the solution to pH 3 and precipitate out the soluble keratin derivative;
(v) The soluble keratin derivative was collected by filtration and washed with water before freeze drying to give sample ‘SPD’.

The above method was repeated on three other occasions following the same procedure but using less succinic anhydride to give samples: 4.15 g (SPC), 2.075 g (SPB) and 1 g (SPA) of succinic anhydride. The samples were then analyzed to determine the extent of the reaction.

The amount of soluble keratin derivative present in the samples was determined using an ashing method. Samples were heated to 700° C. and the solid remaining measured as a percentage of the whole solid. The samples analyzed gave a soluble keratin derivative content of the solids as greater than 99.5% showing that the resulting solid was essentially pure solid keratin derivative.

Infra-red spectra were recorded of all samples as KBr disks on a Perkin-Elmer 2000 FT-IR. Infra red spectra of SPB, SPC and SPD show distinct signals at around 1730 cm⁻¹ due to the carbonyl, showing the presence of the acid group attached to the soluble keratin derivative. The spectrum of SPA shows only a weak carbonyl signal. The degree of substitution (DS) of the soluble keratin derivative is determined by the excess of succinic anhydride used in the reaction. A large excess is needed to gain a high DS.

Primary amines were detected in the soluble keratin derivative using the OPA (ortho-phthaldialdehyde) method of Bertrand-Harb et al. 50 ml of an OPA standard was prepared from 25 ml of 0.1 molL⁻¹ sodium borate, 2.5 ml of 20% SDS, 40 mg of OPA dissolved in 1 ml of MeOH and 100 μL of mercaptoethanol. The volume was made up to 50 ml with water. The reagent was prepared daily and stored in the dark at 25° C. until used. Unknown samples were prepared at a concentration of 2 g/L of protein in 50 mmolL⁻¹ sodium phosphate buffer. 100 μL of each sample was mixed with 2 ml of the OPA standard and incubated for 2 min before the absorbance was recorded at 340 nm. A series of standards were prepared using L-leucine at 0.25 to 2.00 mmolL⁻¹ from which a calibration curve was derived. Table 1 shows the extent of lysine substitution as determined using the OPA method.

TABLE 1
Extent of lysine substitution determined by the OPA method

| | | Degree of |
|---|---|---|
| | Equivalents of succinic | substitution (DS) |
| Sample | anhydride | (%) |
| SPA | 6 | 28 |
| SPB | 12.5 | 74 |
| SPC | 25 | 79 |
| SPD | 50 | 83 |

In succinylation reactions, usually the extent of N-succinylation is higher than O-succinylation due to the instability of O-succinyl tyrosine ester bonds which break rapidly at pH>5.

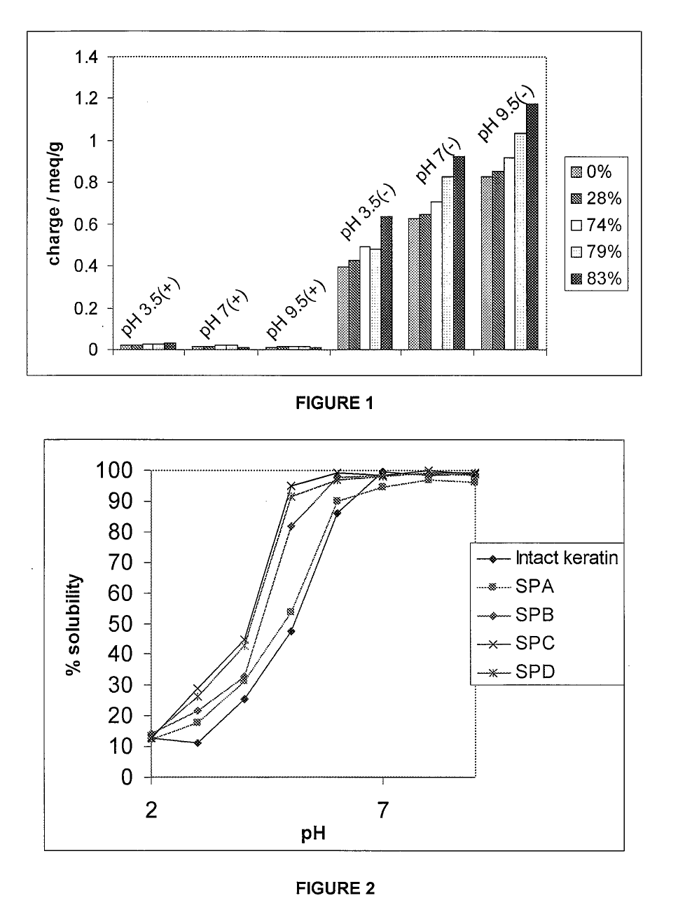

The charge of the molecule was determined using a colloid titration technique. 5 ml of a 0.1% soluble keratin derivative solution was added to a buffer (pH 3.5, 7 or 9.5) and a few drops of toluidine blue and titrated with 1/400N potassium poly(vinyl)sulfate (PVSK) solution to determine the amount of positive charge present in solution. To determine the amount of negative charge, a known amount of 1/400N poly(diallyldimethylammonium)chloride (PDAC) was added to 5 ml of a 0.1% soluble keratin derivative, the buffer (pH 3.5, 7 or 9.5) and a few drops of toluidine blue and back titrated with PVSK. Succinylation is expected to result in a soluble keratin derivative with increased negative charge present and less positive charge as the positively charged lysine groups have been made into negatively charged COO⁻. Colloid titration shows this to be the case with a substantial increase in the amount of negative charge measurable and the amount of positive charge measurable almost undetectable (FIG. 1 and Table 2). The amount of negative charge present is observed to increase with increasing extent of succinylation, showing an increasingly negative species has been generated.

TABLE 2
Amount of charge measured using the colloid titration method.

| | Charge/meq/g | | | | | |
|---|---|---|---|---|---|---|
| | Positive | | | Negative | | |
| Sample | pH 3.5 | pH 7 | pH 9.5 | pH 3.5 | pH 7 | pH 9.5 |
| Intact | 0.0219 | 0.0169 | 0.0113 | 0.396 | 0.625 | 0.826 |
| Keratin | | | | | | |
| SPA | 0.0213 | 0.0169 | 0.0158 | 0.428 | 0.647 | 0.854 |
| SPB | 0.0254 | 0.0238 | 0.0150 | 0.489 | 0.703 | 0.917 |
| SPC | 0.0258 | 0.0207 | 0.0142 | 0.481 | 0.829 | 1.035 |
| SPD | 0.0298 | 0.0125 | 0.0099 | 0.637 | 0.927 | 1.178 |

pH solubility curves were measured by preparing 1% dispersions of the soluble keratin derivative between pH 2 and 10 which were shaken for 1 hour (monitoring the pH every 15 min and adding acid/base where necessary), the solid was filtered off dried and weighed to determine the amount of soluble keratin derivative which had dissolved at a set pH. Plots of pH vs. % solubility allowed an estimation of the isoelectric point or pI and the effect of the chemical modification on the pI. pH solubility curves (FIG. 2) show a steady increase in solubility in acidic pH with increasing DS. This is caused by the addition of negatively charged groups causing the pI for the molecule to shift to lower pH thus increasing the solubility above that pH.

The emission spectra for the soluble keratin derivative samples were recorded using a Hitachi F-4000 fluorescence spectrophotometer. The excitation wavelength used was 340 nm, and the excitation and emission bandpass were both 5 nm. Samples were 0.01% in water. The emission maxima for the succinylated proteins are presented in Table 3.

TABLE 3
λₘₐₓ for emission spectra of proteins

| | |
|---|---|
| Sample | Wavelength/nm |

| | | |
|---|---|---|
| Intact Keratin | 337.6 | |
| SPA | 340.0 | |
| SPB | 341.8 | 364.0 (sh) |
| SPC | 342.2 | 365.6 (sh) |
| SPD | 344.0 | 369.8 (sh) |

Sample SPA with a lower DS shows a slight change in its emission maximum with the maximum red shifting to 340.0 nm. Increasing succinylation results in a larger red shift of the emission maxima and a new shoulder growing in at 369.8 in the case of sample SPD. The introduction of the bulky negatively charged succinyl groups has resulted in the exposure of more tryptophan to a polar environment perhaps by forced unfolding of the soluble keratin derivative due to unfavorable charge repulsions.

Further experiments were completed by the inventors using the above methodology but using hydrolyzed keratin protein as the base protein material rather than intact protein. In this case, the results found regarding changed charge and substitution was comparable.

The results show that succinylation of keratin protein results in a keratin derivative with increased negative charge present and with different characteristics compared to the starting keratin protein. Succinylated keratin derivatives show a lowered pI with an increased positive charge compared to a non-derived keratin protein.

### Example 2

### Manufacturing a Quaternised Keratin Derivative

This Example describes investigations into the derivatization of soluble keratin proteins. It describes the procedures by which the soluble keratin proteins are quaternised.

Quaternisation of the soluble keratin protein was performed by addition of a positively charged quaternary ammonium salt to the lysine groups and terminal amine group in the soluble keratin protein. This reaction was found to be repeatable with compounds with the same properties generated each time the experiment was performed under the same conditions. More specifically, quaternisation of soluble keratin protein was performed using the following method:
(i) To 4 Schott bottles containing 40.25 g of an intact soluble keratin solution (3.2%, pH=7.57, each bottle contained 1.25 g of protein) was added glycidyl trimethyl ammonium chloride in varying amounts (0.625 ml (0.5 g) in QuatA, 1.25 ml (1 g) in QuatB, 2.5 ml (2 g) in QuatC and 5 ml (4 g) in QuatD).
(ii) The bottles were sealed and shaken well before being placed in a preheated incubator-shaker at 40° C. for 18 hours.
(iii) After 18 hours the samples were removed from the incubator and dialyzed before being freeze dried.

The samples produced were then analyzed using the same methods as described above for succinylation to determine the extent of the quaternisation reaction. The results found from the analysis follow below.

After dialyzing the samples (QuatA-D) were found to be greater than 99% soluble keratin derivative by ashing except for QuatA which was observed to be 96% soluble keratin derivative. Infra red spectra measured for each samples (Quat A-D) showed no discernable difference to the spectrum of intact keratin as the substitution did not involve any strongly Infra red active signals. The degree of substitution (DS) of the soluble keratin derivative is determined by the amount of glycidyl trimethyl ammonium chloride (GTMAC) used in the reaction. Table 4 shows the extent of lysine substitution as determined using the OPA method.

TABLE 4
Extent of lysine substitution determined by the OPA method

| | Amount of GTMAC | Degree of |
|---|---|---|
| Sample | added (ml) | substitution DS (%) |
| QuatA | 0.625 | 7 |
| QuatB | 1.25 | 41 |
| QuatC | 2.5 | 65 |
| QuatD | 5 | 85 |

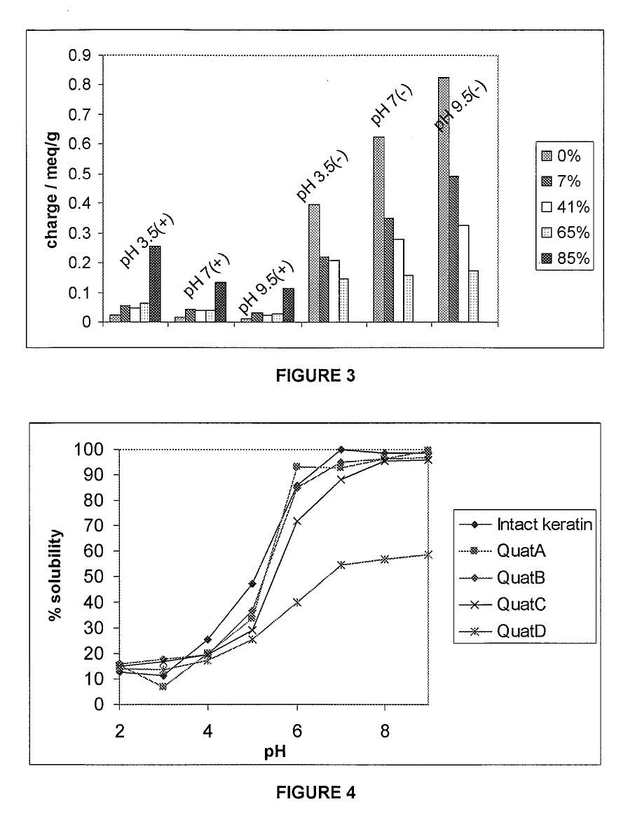

The charge of the QuatA-D samples was determined using a colloid titration technique. This technique uses the reaction between positively charged polyelectrolytes and negatively charged polyelectrolytes to determine the amount of charge present in an unknown. The negative polyelectrolyte used, potassium poly(vinyl)sulfate (PVSK) interacts with toluidine blue giving a red-violet colored solution thus positively charged species maybe directly titrated for with PVSK until the blue solution goes red-violet. Negatively charged species need to have a known amount of the positively charged polyelectrolyte poly(diallyldimethylammonium)chloride (PDAC) added to the solution and then back titrate with PVSK. The titrations for soluble keratin derivative need to be repeated at several pH levels to allow for the ionizable groups. The technique is also dependant on the polyelectrolytes being able to access all charge within the molecule. In the case of soluble keratin derivative, the folding experienced by the soluble keratin derivative may result in some of the charge being strongly bound to other parts of the soluble keratin derivative and thus not being available in the titration. Titrations performed on intact keratin show that only small amounts of positive charge are detectable which decrease with increasing pH while a factor of approximately 10 times more negative charge is detectable the amount of which increases as expected with increasing pH. It is known for intact keratin that this soluble keratin derivative is negative in character as the cysteine groups are all S-sulfonates which are negatively charged from a low pH. On performing titrations for Quat A-D, it is evident that the amount of positive charge has increased slightly in the case of A-C and extensively for D while the amount of negative charge has decreased significantly (Table 5 and FIG. 3). The amount of negative charge present in the sample should not have been affected by the chemical reaction and therefore the decrease in negative charge is attributed to the increased amount of positive charge present binding the negative species. In the case of QuatD it is observed that no negatively charged species are detected. The degree of substitution of the lysine in this sample is only slightly greater than the degree of substitution in C nevertheless the behavior is significantly altered. It is possible that with such a large excess other amino acids may have reacted. There is also the possibility of unreacted GTMAC still being present in solution although this is unlikely due to the dialysis treatment the sample undergoes.

TABLE 5
Amount of charge determined in the quaternised
samples by the colloid titration method.

| | Charge/meq/g | | | | | |
|---|---|---|---|---|---|---|
| | Positive | | | Negative | | |
| Sample | pH 3.5 | pH 7 | pH 9.5 | pH 3.5 | pH 7 | pH 9.5 |
| Intact | 0.0219 | 0.0169 | 0.0113 | 0.396 | 0.625 | 0.826 |
| keratin | | | | | | |
| QuatA | 0.0541 | 0.0429 | 0.0317 | 0.221 | 0.350 | 0.493 |
| QuatB | 0.0481 | 0.0373 | 0.0247 | 0.210 | 0.279 | 0.327 |
| QuatC | 0.0611 | 0.0401 | 0.0265 | 0.147 | 0.157 | 0.173 |
| QuatD | 0.255 | 0.135 | 0.113 | 0 | 0 | 0 |

The solubility of a soluble keratin derivative at differing pH is partly dependant on the number of ionized groups present at that pH. The soluble keratin derivative will be least soluble around its ionization point (pI) as at this pH the over all charge of the molecule will be neutral. These samples were found to have decreased solubility in acidic mediums when compared with intact keratin with the solubility strongly dependant on the degree of substitution. Sample D (85% substituted) was observed to largely precipitate out during dialysis at pH 7. FIG. 4 shows the pH-solubility curves for intact keratin and the four quaternised samples, Quat A-D. It is obvious from this plot that the solubility of the sample decreases at lower pH with increasing quaternisation. Sample QuatD is found to be very insoluble only achieving 60% solubility at pH 9. This lack of solubility in QuatD is probably due to self aggregation as there is now a significant amount of positive charge present to associate with the negative charge. These results suggest as expected the pI is shifting to higher pH with increasing DS.

The λₘₐₓ for the emission spectra of intact keratin and the quaternised samples Quat A-D are shown in Table 6. The spectrum of intact keratin has a maximum at 338.0 nm. These shift very little for Quat A and B while for Quat C and D a slight shift to shorter wavelength is seen, meaning with increasing positive charge in the molecule a blue shift is observed. It is thought that the exposure of tryptophan residues to a more polar environment causes the emission to red shift, therefore a blue shift may arise due to the emissive amino acids experiencing a less polar environment. The increase in positive charge may be encouraging the protein to fold more tightly instead of the repulsive effect that was experienced previously.

TABLE 6
λₘₐₓ for emission spectra of proteins.

| Sample | Wavelength/nm |
|---|---|
| Intact keratin | 338.0 |
| QuatA | 336.2 |
| QuatB | 335.8 |
| QuatC | 333.8 |
| QuatD | 332.4 |

The above trial used intact keratin to form the derivative. A further soluble keratin derivative was produced (termed QuatP) which used hydrolyzed keratin. The QuatP solution was manufactured by the steps of:
(i) 250 ml of a 15.1% solution of unmodified peptide was placed into a 500 ml Schott bottle.
(ii) The pH was adjusted to 9 as this should maximize the amount of free amine groups available to react with. 12.5 ml of GTMAC (glycidyl trimethyl ammonium chloride) was added and the bottle was shaken well and sealed with parafilm. It was placed in a preheated shaking water bath at 40° C. and 120 rpm for 48 hours.

The successful preparation of the quaternised peptide QuatP was confirmed by the OPA method. The modified peptide was found to have less free amino groups than the unmodified peptide (35.77% of the free amino groups had been modified). The final concentration of the modified peptide was calculated to be 14.38% (originally 15.1%). This additional experiment shows that the base protein can be either an intact keratin fraction or a hydrolyzed keratin fraction.

A further trial was undertaken to optimize the quaternisation reaction to understand what influences the degree of substitution and therefore assist in developing the most efficient use of time and reagents. In summary it was found that the degree of substitution increases with both time and the amount of GTMAC added. Therefore, one method of optimizing the process where time is of less concern is to use less reagent and allow the process to run for a longer period of time. Concentration of protein solution was also found to contribute to the degree of substitution. Using a more concentrated protein solution resulted in more substitution occurring. The method of work up for the sample (e.g. dialysis or acid) had no effect on the degree of substitution. The initial pH of the reaction solution was found to have some effect with the optimum pH being approximately 9.

The above results show that quaternisation of the soluble keratin protein results in soluble keratin derivatives with varying degrees of quaternary substitution which show different properties to the starting keratin protein. The results also show that for the quaternised keratin derivatives, the pI has increased and the amount of positive charge present has also increased. In addition, it is shown that the process is repeatable and can be optimized to tailor the degree of substitution required.

### Example 3

### Fatty Acid Substitution

An alternative method is described for chemically modifying soluble keratin protein.

In a first method a fatty acid chloride is used to form a fatty acid keratin derivative (FAP) as shown in Scheme 4 below:

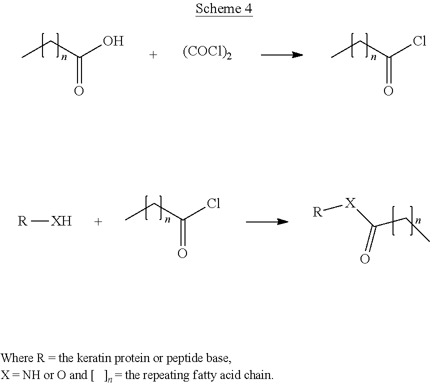

More specifically, reaction of intact, soluble kerartin intermediate filament protein (IFP) with long chain fatty acids to form a first sample (FAP1) was performed using the following method:
(i) To 0.5 g of lauric acid in anhydrous CH₂Cl₂ (10 ml) at 35° C. under N₂ was added 0.41 g of oxalyl chloride dropwise over 10 minutes;
(ii) The reaction mixture was stirred at 35° C. for 2 hours before the solvent was removed under vacuum;
(iii) The resulting solid was dissolved in 10 ml of acetone and added dropwise to either 25 ml or 250 ml of 5% soluble keratin protein solution stirring vigorously in an ice bath at pH 8;
(iv) The pH was maintained at its initial level during the reaction by the addition of 0.1 molL NaOH;
(v) Stirring was continued overnight before the pH was reduced to precipitate the soluble keratin derivative;
(vi) The solid was filtered, washed with acetone to remove any unreacted lauric acid and then freeze dried.

Further samples were produced termed FAP2, FAP3 and FAP4 by varying the amount of lauric acid/oxalyl chloride added and in the case of FAP2, by also lowering the pH to 7. The samples were then analyzed to determine the extent of the reaction.

In a second method, termed ‘EDC coupling’, the intermediate filament protein is reacted with long chain fatty acids using the coupling agent EDC (N-(3-Dimethylaminopropyl)-N′-ethylcarbodiimide hydrochloride) via the process as shown in Scheme 5:

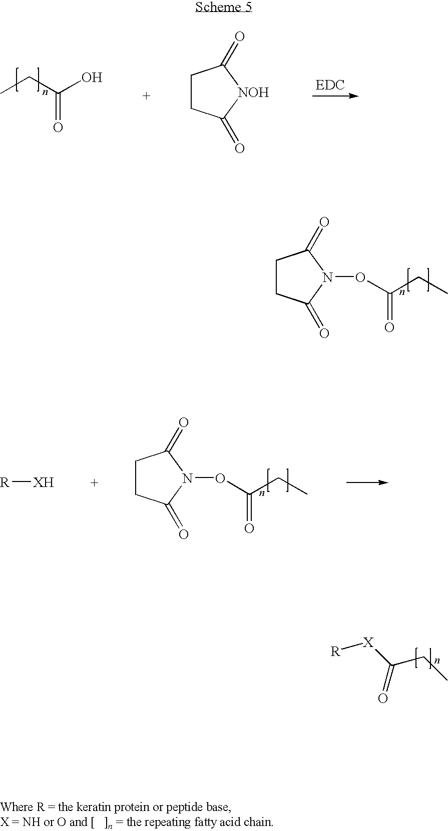

More specifically, the method used to form the EDC product (termed ‘EDCP’) comprises the steps:
(i) 0.1 g of lauric acid, 57 mg of N-hydroxysuccinimide (NHS) in anhydrous ethyl acetate (10 ml) and 0.112 g of EDC were mixed together at room temperature under N₂;
(ii) The reaction mixture was stirred overnight and then filtered to remove the dicyclohexyl urea before the solvent was removed under vacuum;
(iii) The resulting solid was dissolved in 5 ml of THF (tetrahydrofuran) and added dropwise to 50 ml of 5% soluble keratin protein solution containing 5×10⁻⁴ molL⁻¹ sodium bicarbonate;
(iv) The solution was then stirred overnight before the pH was reduced to precipitate the soluble keratin derivative;
(v) The solid was filtered and then freeze dried.

The samples were then analyzed to determine the extent of the reaction.

A summary of the main process variations and the measured extent of lysine substitution determined by the OPA method described in earlier examples is summarized in Table 7 below:

TABLE 7
Extent of lysine substitution determined by OPA method.

| | Amount of lauroyl | pH of | Degree of |
|---|---|---|---|
| | chloride/lauric acid | reaction | substitution |
| Sample | added (equiv) | mixture | DS (%) |
| FAP1 | 1 | 8 | 25 |
| FAP2 | 10 | 7 | 5 |
| FAP3 | 10 | 8 | 47 |
| FAP4 | 50 | 8 | 38 |
| EDCP | 1 | 8 | 30 |

The degree of substitution (DS) of the soluble keratin derivative is largely determined by the amount of lauric acid or lauroyl chloride used in the reaction. As may be appreciated, it is difficult to get 100% substitution of the lysine groups as some are in inaccessible positions, shielded by the folding of the soluble keratin derivative. The amount of substitution achieved for these samples is observed to be less than that achieved with quaternisation and succinylation. This is attributed to the larger size of the lauric acid preventing it from accessing some of the lysine positions. It appears the maximum substitution achievable may be around 50% as increasing the amount of reagents above a 10 fold excess had a negative affect on the extent of the reaction.

Measurement of the hydrophobicity of FAP3 shows that FAP3 is significantly more hydrophobic than that of the unmodified keratin protein.

Further experiments were completed by the inventors using the above methodology but using hydrolyzed keratin protein as the base protein material rather than intact protein. In this case, the results found regarding changed charge and substitution was comparable.

Modifications based on the fatty acid derivatives above comprise using other fatty acids. For example, other fatty acids may be used, particularly those comprising of long chain salts such as O₁₀, C₁₂, O₁₄, C₁₆, C₄₀ or longer In further embodiments, other reagents instead of oxalyl chloride may be used for example, thionyl chloride, inorganic halides and reagents generally with the group COCl.

### Example 4

### Use of Other Keratin Fractions

Intact keratin from the fraction of intermediate filament protein (IFP) is a preferred fraction. As noted earlier in the specification, keratin protein may be divided into other fractions comprising high sulfur proteins (HSP) which are globular proteins found in the matrix of the fiber cortex, as well as in the cuticle and high glycine-tyrosine proteins (HGTP), found mainly in the fiber cortex. It should be appreciated that the present invention is not limited to just the IFP fraction as the HSP and HGTP fractions also have the same amine groups in the protein and the same hydroxyl amino acids.

By way of example, the chemical reaction that would occur for a succinylation process using HSP and HGTP would be as shown in Scheme 6 below:

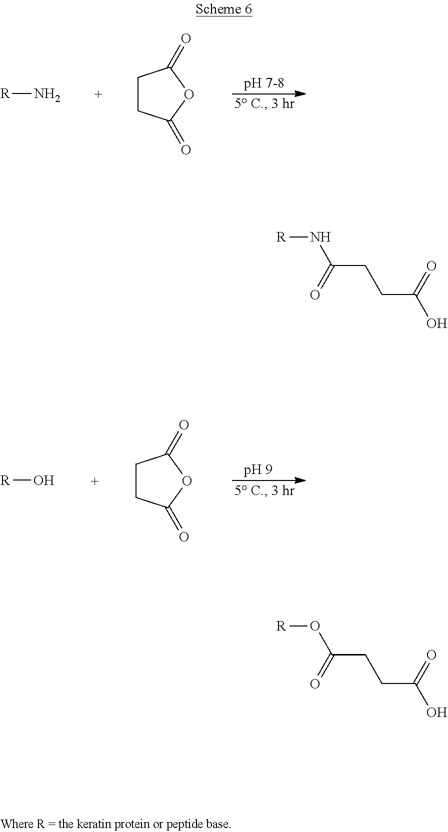

Similarly, the chemical reaction that would occur for a quaternisation process using HSP and HGTP would be as shown in Scheme 7 below:

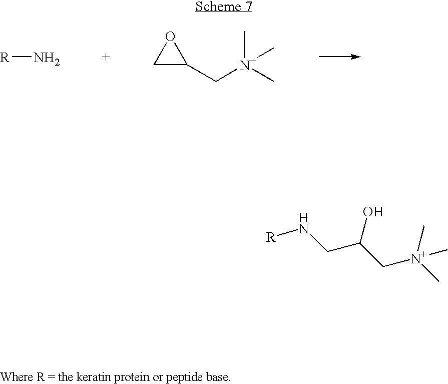

The chemical reaction that would occur for a fatty acid or EDC coupling process using HSP and HGTP would be as shown in Scheme 8 below:

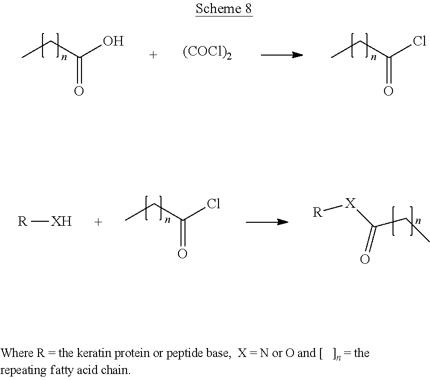

### Example 5

### Surfactant Property Testing

Surfactant properties of the soluble keratin derivatives were tested alongside the non-derivative keratin proteins, such as soluble IFP fractions, and compared to other known measures.

Surface tension measurements were completed using sample SPC and QuatC described in Example 1 and Example 2, respectively. As shown in Table 8 below, the surface tension reducing properties of the soluble keratin derivative compounds was comparable to mildly contaminated tap water and the non-derivatized keratin. Surprisingly, only half the concentration of non-derivatized keratin was required to achieve the same surface tension reducing effect.

TABLE 8
Surface tension measurements:

| | Concentration (g/L) | Value |
|---|---|---|
| Intact non-derivatised | 10 | 43.2 dynes · cm⁻¹ |
| IFP fraction keratin | | |
| SPC | 5 | 48.6 dynes · cm⁻¹ |
| QuatC | 5 | 46.8 dynes · cm⁻¹ |
| Ethanol | | 22.8 dynes · cm⁻¹ |
| Mildly Contaminated | | 51.5 dynes · cm⁻¹ |
| Water | | |
| Reverse-osmosis water | | 72.3 dynes · cm⁻¹ |
| Double distilled water | | 72.3 dynes · cm⁻¹ |

Foaming experiments were also performed to test the foam height produced and the time that the foam remained intact before collapse. As shown in Table 9 below, the soluble keratin derivatives performed substantially better than non-derivatized protein in terms of foaming and time to collapse. Surprisingly, the concentration of the soluble keratin derivatives was also substantially less than for the non-derivatized keratin to achieve the same effect.

TABLE 9
Foaming experiments

| | Concentration | Foam height (zero | Time of total foam |
|---|---|---|---|
| | (g/L) | time) | collapse |
| Intact non- | 1.0 | 0 cm | 0 seconds |
| derivatized IFP | 10 | 1.0 cm | 3 minutes |
| fraction keratin | 50 | 3.0 cm | 14 minutes |
| SPC | 50 | 3.9 cm | 14 minutes |
| | 10 | 2 cm | 16 minutes |
| QuatC | 50 | 3.4 cm | 24 minutes |
| | 10 | 3.2 cm | 20 minutes |

Further trials were completed to test the emulsification effects of the soluble keratin derivativeS by formation of water in oil (w/o) emulsions. The method comprised the steps of shaking 15 ml of either soybean cooking oil (sample 1) or 15 ml of castor oil (sample 2) with 15 ml of water in the presence of 10 ml of soluble keratin derivative. The dispersions were then left to stand for approximately 1 minute and subsequently examined to check for the presence or otherwise of an emulsion. In both samples, water in oil (w/o) emulsions were formed indicating that the soluble keratin derivatives of the present invention can act as emulsifiers and therefore have useful surfactant properties.

To summarize, the soluble keratin derivatives show surfactant properties. In addition, these properties show a significant difference to non-derivatized keratin.

### Example 6

### Personal Care Products and Formulations Containing Derivatized Keratin

Examples are now provided of various personal care products using the soluble keratin derivatives of the present invention. It should be appreciated that due to the multiple beneficial properties, the soluble keratin derivative are well suited to use in personal care products. For example, the soluble keratin derivative have the ability to bind to the skin and trap moisture in the skin therefore moisturizing the skin. As should be appreciated from later examples in this specification, the soluble keratin derivative properties are also useful in hair products as use of the soluble keratin derivative makes hair management easier through reduced combing force and improved ‘feel’. The examples below are provided by way of illustration only and should not be seen as limiting.

In each formulation ‘keratin derivative’ is included at an indicative level. Keratin derivative refers to keratin proteins that have been modified to include either a positive or a negative region, using methods comprising those described above. Unless otherwise stated, it is convenient to provide the keratin derivative in the form of a dilute aqueous solution and include the appropriate amount of this solution in the formulation to achieve the keratin derivative level indicated. Percentages are expressed as w/v.

Conditioning shampoo

| | |
|---|---|
| Sodium lauryl sulphate 28% | 25.0% |
| Sodium laureth-2-sulphate 70% | 4.0 |
| Cocamide DEA 70% | 3.5 |
| Cocamidopropyl betaine (30%) | 3.0 |
| Keratin derivative | 0.5 |
| Sodium chloride | q.s. |
| Citric acid | q.s. |
| Fragrance | q.s. |
| Preservative | q.s. |
| Water | q.s. to 100 |

Procedure: Combine 35.0 g water, sodium laureth sulphate and sodium lauryl sulphate. Heat to 65° C. until dissolved. Add cocamide DEA and allow to cool. Mix betaine with water and add to phase A. Add keratin derivative, adjust the pH to 6.5 with citric acid. Add preservative and fragrance as required, adjust to desired thickness with sodium chloride and add remaining water.

Hair gel

| | |
|---|---|
| Carbomer (Carbopol Ultrez 10) | 0.5% |
| Disodium EDTA | 0.05 |
| Glycerin | 4.0 |
| Triethanolamine (20%) | 3.0 |
| Keratin derivative | 0.45 |
| Preservative | q.s. |
| Fragrance | q.s. |
| Water | q.s. to 100 |

Procedure: Heat 60.0 g of water to 70° C. and add to carbopol, EDTA and glycerol. Mix vigorously. Cool. Add triethanolamine to adjust pH to 6.3. Add keratin derivative. Combine preservative and remaining water and add. Mix thoroughly and add fragrance as desired.

Clear Body/Facial Cleanser and Shampoo

| | |
|---|---|
| Ammonium lauryl sulphate 28% | 25.0% |
| Disodium laureth sulfosuccinate | 20.0 |
| Cocamidopropyl betaine | 8.0 |
| Keratin derivative | 0.5 |
| Sodium chloride | q.s. |
| Fragrance (parfum) | q.s. |
| Preservative | q.s. |
| Water (aqua) | q.s. to 100 |

Hair Conditioner

| | |
|---|---|
| Cetrimonium chloride | 5.0% |
| Stearyl alcohol | 4.5 |
| Keratin derivative | 0.25 |
| Fragrance | q.s. |
| Preservative | q.s. |
| Water | q.s. to 100 |

Hair Mousse

| | |
|---|---|
| Keratin derivative | 0.25% |
| Hydrogenated tallow trimonium chloride | 0.20 |
| Nonoxynol-10 | 0.35 |
| Alcohol | 10.0 |
| Butane-48 | 10.0 |
| Water | q.s. to 100 |

Setting lotion

| | |
|---|---|
| Carbomer (Carbopol Ultrez 10) | 2.0% |
| Mineral oil (light) | 0.20 |
| Keratin derivative | 0.25 |
| Alcohol | 37.5 |
| Fragrance | q.s. |
| Water | q.s. to 100 |

Hairspray

| | |
|---|---|
| VA/Crotonates/Vinyl Neodeconoate Copolymer | 1.60% |
| (Resyn 28-2930) | |
| Aminomethyl propanol | 0.15 |
| PEG-75 lanolin | 0.20 |
| Keratin derivative | 0.25 |
| Alcohol | 65.05 |
| Butane 30 | 28.0 |

Pre-perming solution

| | |
|---|---|
| TEA lauryl sulphate | 30.0% |
| Cocamidopropyl dimethylamine oxide | 10.0 |
| Cocamide DEA | 7.5 |
| Cocamidopropyl betaine | 20.0 |
| Cocamide MEA | 3.0 |
| Keratin derivative | 0.5 |
| Fragrance | q.s. |
| Preservative | q.s. |
| Water | q.s. |

Post-perming solution

| | |
|---|---|
| Keratin derivative | 0.5% |
| Cocamidopropyl dimethylamine oxide | 10.0 |
| PPG-5-ceteth-10-phosphate | 0.5 |
| Glycerin | 3.0 |
| Hydroxypropyl methylcellulose | 1.5 |
| Fragrance | q.s. |
| Preservative | q.s. |
| Water | q.s. to 100 |

Moisturizing cream

| | |
|---|---|
| Cetearyl alcohol and ceteareth-20 | 5.0% |
| Cetearyl Alcohol | 2.0 |
| Mineral oil (light) | 5.0 |
| Keratin derivative | 0.5 |
| Preservative | 0.3 |
| Fragrance | q.s. |
| Water | q.s. to 100 |

Hand and Body Lotion

| | |
|---|---|
| Polyglyceryl-3 methylglucose distearate | 4.0% |
| Stearyl/behenyl beeswaxate | 3.0 |
| Octyldodecanol | 4.0 |
| Avocado oil | 6.0 |
| Mineral oil | 3.0 |
| Jojoba oil | 2.0 |
| Keratin derivative | 0.5 |
| Ceramide III | 0.2 |
| Propylene glycol | 3.0 |
| Preservative | q.s. |
| Fragrance (Parfum) | q.s. |
| Water (aqua) | q.s. to 100 |

Anti-Wrinkle Treatment Cream

| | |
|---|---|
| Sodium behenoyl lactylate | 2.0% |
| Cetearyl alcohol | 3.0 |
| Glyceryl stearate | 2.6 |
| Isopropyl palmitate | 6.0 |
| Sunflower seed oil | 6.0 |
| Keratin derivative | 0.5 |
| Glycerine | 3.0 |
| Magnesium ascorbyl phosphate (and) lecithin | 6.0 |
| (Rovisome-C, R.I.T.A) | |
| Preservative | q.s. |
| Water | q.s. to 100 |

Facial Moisture Cream

| | |
|---|---|
| Myristyl lactate | 3.0% |
| Laneth-25 (and) ceteth-25 (and) oleth-25 (and) | 1.0 |
| Steareth-25 (Solulan 25, Amerchol) | |
| Mineral oil (70 visc.) | 16.5 |
| Petrolatum | 3.0 |
| Tocotrienol | 1.0 |
| Carbomer 934 | 0.75 |
| Keratin derivative | 0.5 |
| Triethanolamine (10% aq.) | 7.5 |
| Preservative | q.s. |
| Fragrance | q.s. |
| Water | q.s. to 100 |

Moisturizing Body Lotion

| | |
|---|---|
| Methyl glucose dioleate | 2.0% |
| Methyl glucose sesquistearate | 1.5 |
| Methyl gluceth-20 distearate | 1.5 |
| Cetearyl alcohol (and) ceteareth-20 | 1.5 |
| Isopropyl palmitate | 3.0 |
| Ceramide 3, hexyldecanol | 2.0 |
| Methyl gluceth-10 | 3.0 |
| Keratin derivative | 0.5 |
| Carbomer 1342 | 0.2 |
| Triethanolamine | 0.2 |
| Fragrance | q.s. |
| Preservative | q.s. |
| Water | q.s. to 100 |

Cationic Emollient Lotion

| | |
|---|---|
| Isostearamidopropyl laurylacetodimonium | 5.0% |
| chloride | |
| Lactamide MEA | 3.0 |
| Isostearyl neopentanoate | 15.0 |
| Myristyl myristate | 1.0 |
| Cetyl alcohol | 4.0 |
| Glyceryl isostearate | 3.5 |
| Keratin derivative | 0.5 |
| Preservative | q.s. |
| Water | q.s. to 100 |

Men's facial Conditioner

| | |
|---|---|
| Carbomer (Ultrez 10 Carbopol) | 0.4% |
| Propylene glycol | 1.0 |
| PPG-5-buteth | 0.5 |
| Beta glucan | 2.0 |
| PEG-60 hydrogenated castor oil | 0.5 |
| Triethanolamine (99%) | 0.4 |
| Keratin derivative | 0.5 |
| SD-39 C alcohol (Quantum) | 5.0 |
| Fragrance | q.s. |
| Preservative | q.s. |
| Water | q.s. to 100 |

Moisturizing After Shave Treatment

| | |
|---|---|
| Ceteareth-12 (and) ceteareth-20 (and) cetearyl | 6.0% |
| alcohol (and) cetyl palmitate (and) glyceryl | |
| stearate (Emulgade SE, Henkel) | |
| Cetearyl alcohol | 1.0 |
| Dicaprylyl ether | 8.0 |
| Octyldodecanol | 4.0 |
| Glycerin | 3.0 |
| Carbomer (Ultrez 10 Carbopol) | 0.3 |
| Keratin derivative | 0.5 |
| Bisabolol | 0.2 |
| Ethyl alcohol | 3.0 |
| Water (and) sodium hyaluronate, (and) wheat | 4.0 |
| (Triticum vulgare) germ extract (and) saccharomyces | |
| (and) cerevisiae extract (Eashave, Pentapharm) | |
| Triethanolamine | q.s. |
| Fragrance | q.s. |
| Preservative | q.s. |
| Water | q.s. to 100 |

Antioxidant cream

| | |
|---|---|
| Glycerin (99.7%) | 3.0% |
| Xanthan gum | 0.15 |
| Disodium EDTA | 0.05 |
| Hydrogenated polyisobutene | 1.0 |
| Isopropyl palmitate | 5.0 |
| Petrolatum | 0.75 |
| Dimethicone | 0.75 |
| Cyclopentasiloxane | 3.0 |
| Steareth-2 | 1.0 |
| PEG-100 stearate | 1.9 |
| Cetyl alcohol | 2.0 |
| Ethylhexyl palmitate | 3.0 |
| Polyacrylamide (and) C13-14 isoparaffin (and) | 2.0 |
| laureth-7 (sepigel 305, Seppic) | |
| Keratin derivative | 0.5 |
| Glycerin (and) water (and) Vitis vinifera (grape) | 0.5 |
| seed extract (Collaborative) | |
| Fragrance | q.s. |
| Preservative | q.s. |
| Water | q.s. to 100 |

Liquid detergent

| | |
|---|---|
| Sodium laureth sulphate | 50.0% |
| Cocamide DEA | 3.0 |
| Keratin derivative | 0.25 |
| Sodium chloride | q.s. |
| Preservative | q.s. |
| Citric acid | q.s. |
| Water | q.s. to 100 |

Shower Gel

| | |
|---|---|
| Sodium laureth sulphate | 35.0% |
| Sodium lauroyl sarcosinate | 5.0 |
| Cocoamidopropyl betaine | 10.0 |
| Cocoamidopropyl hydroxyl sultaine | 5.0 |
| Glycerine | 2.0 |
| Keratin derivative | 0.15 |
| Tetrasodium EDTA | 0.25 |
| Citric acid | q.s. |
| Fragrance | q.s. |
| Preservative | q.s. |
| Water | q.s. to 100 |

Foaming bath gel

| | |
|---|---|
| TEA lauryl sulphate | 40.0% |
| Lauroyl diethanolamide | 10.0 |
| Linoleic diethanolamide | 7.0 |
| PEG-75 lanolin oil | 5.0 |
| Keratin derivative | 0.25 |
| Tetrasodium EDTA | 0.5 |
| Fragrance | q.s. |
| Preservative | q.s. |
| Dyes | q.s. |
| Water | q.s. to 100 |

Nail Polish - First coat

| | |
|---|---|
| Keratin derivative | 10.0% |
| Sodium hydroxide (4%) | 10.0 |
| Keratin fraction (SHSP or SPEP) | q.s. |
| Sodium lauryl sulphate | q.s. |
| Dye or Pigment | q.s. |
| Water | q.s. to 100 |

Nail Glosser

| | |
|---|---|
| Keratin derivative | 10.0% |
| Keratin fraction (SHSP or sulfonated keratin peptide) | q.s. |
| Sodium hydroxide (4%) | 10.0 |
| Sodium lauryl sulphate | q.s. |
| Water | q.s. to 100 |

Mascara

| | |
|---|---|
| PEG-8 | 3.0% |
| Xanthan gum | 0.50 |
| Tetrahydroxypropyl ethylenediamine | 1.3 |
| Carnauba wax | 8.0 |
| Beeswax | 4.0 |
| Isoeicosane | 4.0 |
| Polyisobutene | 4.0 |
| Stearic acid | 5.0 |
| Glyceryl stearate | 1.0 |
| Keratin derivative | 0.25 |
| Pigments | 10.0 |
| Polyurethane-1 | 8.0 |
| VP/VA Copolymer | 2.0 |
| Preservative | q.s. |
| Fragrance | q.s. |
| Water | q.s. to 100 |

Liquid Foundation

| | |
|---|---|
| Polysorbate 80 | 0.1% |
| Potassium hydroxide | 0.98 |
| Keratin derivative | 0.25 |
| Titanium dioxide/talc, 80% | 0.1 |
| Talc | 3.76 |
| Yellow iron oxide/talc, 80% | 0.8 |
| Red iron oxide/talc, 80% | 0.38 |
| Black iron oxide/talc, 80% | 0.06 |
| Propylene glycol | 6.0 |
| Magnesium aluminum silicate | 1.0 |
| Cellulose gum | 0.12 |
| di-PPG-3 myristyl ether adipate | 12.0 |
| Cetearyl alcohol (and) ceteth-20 phosphate (and) | 3.0 |
| dicetyl phosphate (Crodafos CS 20 Acid) | |
| Steareth-10 | 2.0 |
| Cetyl alcohol | 0.62 |
| Steareth-2 | 0.5 |
| Preservative | q.s. |
| Water | q.s. to 100 |

Shaving Cream

| | |
|---|---|
| Sodium cocosulfate | 5.0% |
| Keratin derivative | 0.25 |
| Glycerin | 7.0 |
| Disodium lauryl sulfosuccinate | 50.0 |
| Disodium EDTA | q.s. |
| Sodium chloride | q.s. |
| Citric acid | q.s. |
| Fragrance | q.s. |
| Preservative | q.s. |
| Water | q.s. to 100 |

Lipstick

| | |
|---|---|
| Octyldodecanol | 22.0% |
| Oleyl alcohol | 8.0 |
| Keratin derivative | 0.16 |
| C30-45 alkyl methicone | 20.0 |
| Lanolin oil | 14.0 |
| Petrolatum | 5.0 |
| Bentone 36 (Rheox) | 0.6 |
| Tenox 20 (Eastman) | 0.1 |
| Pigment/castor oil | 10.0 |
| Preservative | q.s. |
| Cyclomethicone | q.s. to 100 |

Sulfite Hair Straightener

| | |
|---|---|
| Carbomer (Carbopol 940) | 1.5% |
| Ammonium bisulphate | 9.0 |
| Diethylene urea | 10.0 |
| Cetearth 20 | 2.0 |
| Keratin derivative | 0.5 |
| Fragrance | q.s. |
| Ammonium hydroxide 28% | q.s. to pH 7.2 |
| Water | q.s. to 100 |

Post straightening neutralizing solution

| | |
|---|---|
| Sodium bicarbonate | 2.35% |
| Sodium carbonate | 2.94 |
| EDTA | 0.15 |
| Cetearth 20 | 0.2 |
| Keratin derivative | 0.5 |
| Fragrance | q.s. |
| Water | q.s. to 100 |

Pre-relaxer Conditioner

| | |
|---|---|
| Cationic polyamine | 2.0% |
| Imidazolidinyl urea | 0.25 |
| Keratin derivative | 0.5 |
| Fragrance | q.s. |
| Preservative | q.s. |
| Water | q.s. to 100 |

Alkali Metal Hydroxide Straightener (Lye)

| | |
|---|---|
| Bentonite | 1.0% |
| Sodium Lauryl Sulphate | 1.5 |
| PEG-75 lanolin | 1.5 |
| Petrolatum | 12.0 |
| Cetearyl alcohol | 12.0 |
| Sodium hydroxide | 3.1 |
| Keratin derivative | 0.5 |
| Fragrance | q.s. |
| Water | q.s. to 100 |

Post Relaxing Shampoo

| | |
|---|---|
| Sodium lauryl sulphate | 10.0% |
| Cocamide DEA | 3.0 |
| EDTA | 0.2 |
| Keratin derivative | 0.5 |
| Citric acid | q.s. to pH 5.0 |
| Fragrance | q.s. |
| Preservative | q.s. |
| Water | q.s. to 100 |

Hair tonic/cuticle cover

| | |
|---|---|
| Glycerine | 5.5% |
| EDTA | 0.07 |
| Carbomer (Carbopol Ultrez 10) | 0.33 |
| Triethanolamine (20%) | 1.0 |
| Keratin derivative | 0.5 |
| Ethanol | 10.0 |
| Preservative | q.s. |
| Water | q.s. to 100 |

Leave in hair conditioner

| | |
|---|---|
| Cetyl alcohol | 5.0% |
| Glyceryl stearate | 3.0 |
| Petrolatum | 0.7 |
| Isopropyl myristate | 1.5 |
| Polysorbate 60 | 1.0 |
| Dimethiconol & cyclomethicone | 4.0 |
| Glycerine | 7.0 |
| EDTA | 0.1 |
| D-panthenol | 0.2 |
| Keratin derivative | 0.5 |
| Cyclomethicone | 4.0 |
| Fragrance | q.s. |
| Preservative | q.s. |
| Water | q.s. to 100 |

Post Hair-dyeing Conditioner

| | |
|---|---|
| Quaternium-40 | 2.0% |
| Keratin derivative | 0.5 |
| Amphoteric-2 | 4.0 |
| Hydroxyethyl cellulose | 2.0 |
| Phosphoric acid | q.s. to pH 4.5 |
| Fragrance | q.s. |
| Water | q.s. to 100 |

Temporary Hair Coloring Styling Gel

| | |
|---|---|
| Dimethicone copolyol | 1.5% |
| PPG-10 methyl glucose ether | 1.0 |
| Polyvinylpyrrolidone | 2.5 |
| Triisopropanolamine | 1.1 |
| Carbomer (Carbopol 940) | 0.6 |
| Laureth-23 | 1.0 |
| Phenoxyethanol | 0.2 |
| Keratin derivative | 0.5 |
| EDTA | 0.01 |
| D&C orange 4 | 0.12 |
| Ext D&C Violet 2 | 0.02 |
| FD&C yellow 6 | 0.02 |
| Ethanol | 5.0 |
| Fragrance | q.s. |
| Water | q.s. to 100 |

### Example 7

### Influence of Succinylation on Hair Physical Properties

A trial was completed to determine the physical condition of hair tresses following repeated washing with a succinylated keratin derivative containing solution compared with industry standards such as sodium laureth sulphate (SLES). Scanning Electron Microscopy (SEM) and TLC analysis were performed to explore the changes in the surface morphology and lipid content of the hair fibers due to the different treatments preformed on the tresses.

Six hair tresses were made by weighing approximately 1.5 g of natural red hair and fixing the hair into tresses with a tie. The tresses were pre-treated by washing with a 2% sodium laureth sulfate (SLES) solution (prepared from 70% SLES and diluted to achieve 2% solution) for 2 minutes and rinsed thoroughly (until no bubbles, no surfactant left) with warm water (˜40° C.) for 2 more minutes. Next the hair tresses were dried in air.

Different washing treatments were then performed on the hair tresses using the following methodologies (each washing treatment completed twice):
SLES washing treatment: Hair was washed using SLES for a period of one week. Washing was completed by placing the hair tress into a 5% SLES solution for 1 hour in a rocking table, after this the hair was rinsed thoroughly with water warm water (˜40° C.) for 2 minutes (until no bubbles, no surfactant left) and then dried in air. This washing process was performed twice every day, giving a total of 10 washes.
Keratin derivate washing treatment: Hair was washed in succinylated keratin derivative (termed sample ‘SPC’ in earlier Examples) for period of one week. The washing process was completed as described above whereby the hair tress was placed in a 5% succinylated keratin derivative solution for 1 hour in a rocking table, after this the hair was rinsed thoroughly with water warm water (˜40° C.) for 2 minutes (until no bubbles, no surfactant left) and then dried in air. This washing process was performed twice every day, giving a total of 10 washes.

After washing the hair samples were obtained in duplicate labeled: (A, B) SLES washed hair; (C, D) SPC washed hair; (E, F) untreated hair.

### Scanning Electron Microscope (SEM) Analysis

An SEM study was performed to all hair samples (A to F), to evaluate the possible changes on the surface morphology of the hair fibers due to the different treatments made.

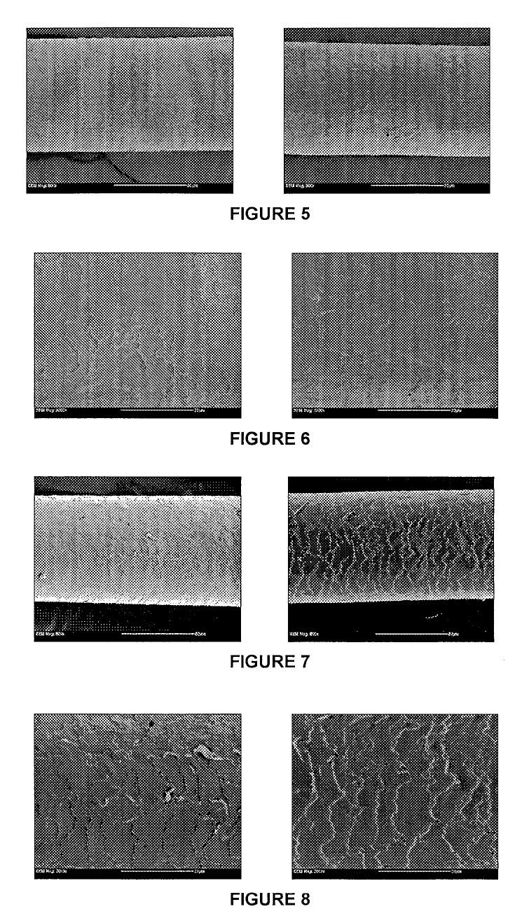

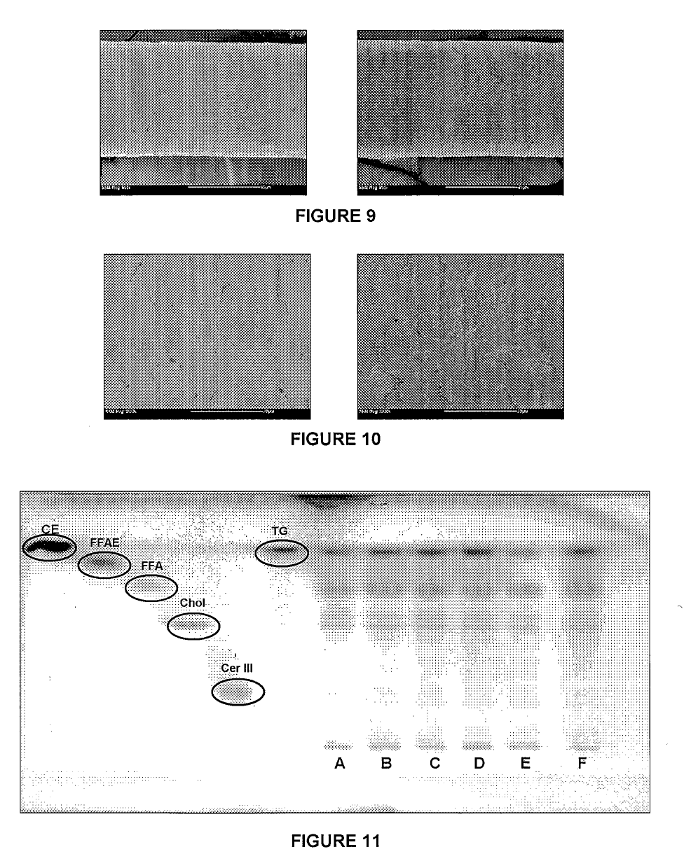

For this, the hair sample was mounted onto 10 mm brass stubs using conductive carbon adhesive tape and sputter coated from a gold/palladium source. Coating thickness was ˜200 Angstroms. Samples were studied using a Jeol JSM 6100 Scanning Electron Microscope. The microscope was operated at 7.0 kV and samples viewed at a working distance of 15 mm. 10 fibers of each hair sample were viewed and representative images taken. Images obtained are shown in FIGS. 5-10.

The resulting images showed that sample A (SLES washed hair) shows the most damage to the cuticle of all the samples, indicating that SLES washing process is the one that causes the most damage to the surface of the hair. This damage, specifically cuticle lifting, can occur as products are washed off the surface of the hair. Less damage was observed for SPC treated hair.

Results also showed that residue is present on all samples but, as expected, the untreated hair samples (Samples E and F) showed the least residue. The largest residue was observed on the hair samples washed with the keratin derivative solution SPC (Samples C and D). Cuticle detail is obscured in areas on these samples suggesting a relatively persistent layer of surfactant protein protecting the cuticle.

### Lipid Extraction Analysis

The lipids of all hair samples (A to F) were Soxhlet extracted with 200 ml of chloroform/methanol (2:1) azeotrope for 7 hours and finally were immersed in the chloroform/methanol mixture overnight. The different extracts were then concentrated and dissolved in 10 ml of chloroform-methanol (2:1) prior to analysis. After extraction three extracts resulted being (in duplicate): (A,B) extract from SLES washed hair; (C,D) extract from SPC washed hair; (E,F) extract from untreated hair.

As shown in Table 10 below, washed hair samples (both the SLES and SPC treatments), give lower levels of lipids extracted when compared to the amount of lipids extracted from the untreated hair samples. No differences were found in the amount of lipid extracted between the two different washing treatments.

TABLE 10
Percentage of Lipids Extracted from the different Hair Samples

| Hair Sample | 1 | 2 | Mean |
|---|---|---|---|
| Untreated | 3.47 | 4.26 | 3.86 |
| SLES | 2.91 | 3.59 | 3.25 |
| Washed | | | |
| SPC Washed | 3.50 | 3.04 | 3.27 |

### Lipid Analysis

The total amount of lipids extracted was further analyzed by drying the extracts under a flow of N₂ until they reached a constant weight. Each extract was qualitatively analyzed by thin-layer chromatography with the following solvent system: ether, pet ether 40-60, acetic 100:97:3. The spots were detected with a 10% CuSO₄/8% H₃PO₄ solution by immersing the TLC plate in the solution for 10 seconds and then heating it at 180° C. for 10 minutes.

The results for the thin-layer chromatography analysis of the lipids of the different hair samples are shown in FIG. 11. The results indicate that slight differences in the amount of certain classes of lipids can be found for the different hair samples. Further, these differences are too small to be considered significant, suggesting that the internal hair lipids had not been altered due to the treatments made on the tresses.

### Trial Summary

In this example, the damaging effects of two different washing processes, one using an industry surfactant (SLES) and the other using a succinylated keratin protein derivative (SPC), were compared. Initial treatments of the hair fibers show that both washing methods modify the hair fibers leading to changes in sensorial effects such as softness and smoothness of the treated hair fibers, which appear decreased.

The SEM study demonstrates the differences in the condition of the surface morphology of the hair samples due to the treatments each sample received. Comparing the two different treatments SEM results shows that the SLES treatment is the most damaging and the SPC treatment coats the hair fiber forming a persistent layer of surfactant protein that may act to protect the cuticle.

TLC analysis of the extracts did not show any differences between the different samples indicating that the internal lipids of the hair fibers may have not been altered due to the different treatments made.

### Example 8

### Influence of Hydrolyzed Quaternised Keratin Derivatives on Hair Physical Properties

The aim of this study was to determine the effect of quaternised hydrolyzed keratin derivatives on hair. Hair care formulations with and without keratin derivatives were applied to hair tresses and relevant properties such as combing force (manageability) was measured and compared with the soluble wool keratin peptide and with other polymeric conditioning agents. To support the combing force results, the sensorial properties (softness etc) of hair tresses were evaluated using a panel test. The keratin derivative sample used in this trial was QuatP described above.

Four hair tresses were made up using approximately 3.3 g of hair in each tress.

Each hair tress was washed with a 2% SLES solution (prepared from 70% SLES and diluted to achieve 2% solution) for 2 minutes and rinsed thoroughly (until no bubbles, no surfactant left) with warm water (˜40° C.) for 2 more minutes. Next the hair tresses were dried in air.

After washing the hair tresses and before any treatment, combing force experiments were performed to the hair tresses to eliminate the possible tangles, knots etc and to be sure all tresses have the same initial properties. The tress was pulled upward through the comb and the Force vs. Elongation graph recorded. After completion of the first comb this was repeated for a total of 10 combing strokes for each tress. The number of combings and any difficulties during test (i.e. tangles, knots etc) were recorded.

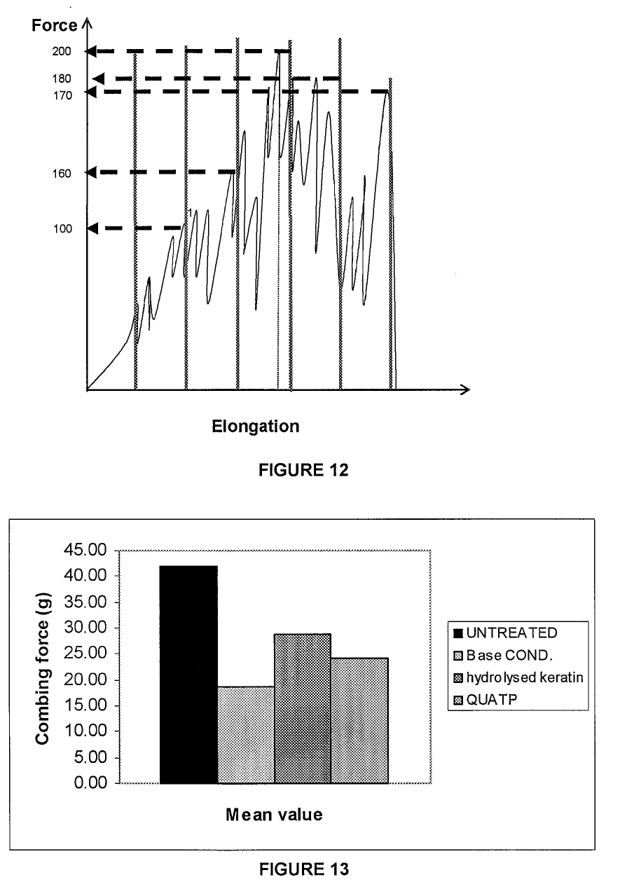

For each force/elongation graph three different parameters were recorded: the average force by making measurements from the first prominent peak to the last prominent peak and sectioning into five equal parts, taking the highest peak in each column and extrapolating to the force axis, as illustrated in FIG. 12, the highest peak graphic and the highest peak given by the Instron. The GeoMean and the percent relative standard deviation was then calculated which was then used to determine the combing forces of the treated hair tresses.

Hair samples were subjected to the following treatments:
Untreated. Sample 1 was kept untreated as a virgin control.
Conditioner base treatment: Sample 2 was wetted with distilled water for 2 minutes. While wet, 3 g of the conditioner base was applied and left on the hair for 2 minutes after which the hair was rinsed thoroughly with warm water (˜40° C.) for 2 minutes. Next the hair was dried in air.
1% Non-derivatised hydrolyzed keratin protein conditioner treatment: hydrolyzed keratin conditioner was made by adding 1.0 g of hydrolyzed keratin and making up to 100.0 g with a conditioner base followed by thorough mixing. Sample 3 was wetted with distilled water for 2 minutes. While wet, 3 g of the conditioner containing 1% hydrolyzed keratin was applied and left on the hair for 2 minutes after which the hair was rinsed thoroughly with warm water (˜40° C.) for 2 minutes. Next the hair was dried in air.
1% Quaternised keratin derivative (termed ‘QUATP’) conditioner treatment: QUATP keratin derivative (made as per the method described in Example 2) conditioner was made by adding 1.0 g of QUATP and making up to 100.0 g with a conditioner base (same as used for the hydrolyzed keratin) followed by thorough mixing. Sample 4 was wetted with distilled water for 2 minutes. While wet, 3 g of the conditioner containing 1% QUATP was applied and left on hair for 2 minutes after which the hair was rinsed thoroughly with warm water (˜40° C.) for 2 minutes. Next the hair was dried in air.

After all hair tresses had been treated and dried, the combing force was measured. Combing force measurements were carried out as described in the pre-treatment combing force measurements part. This was repeated for a total of 10 combing strokes for each tress and the geometric mean was calculated, related to the pre-treatment results and a one tailed student's t-test performed.

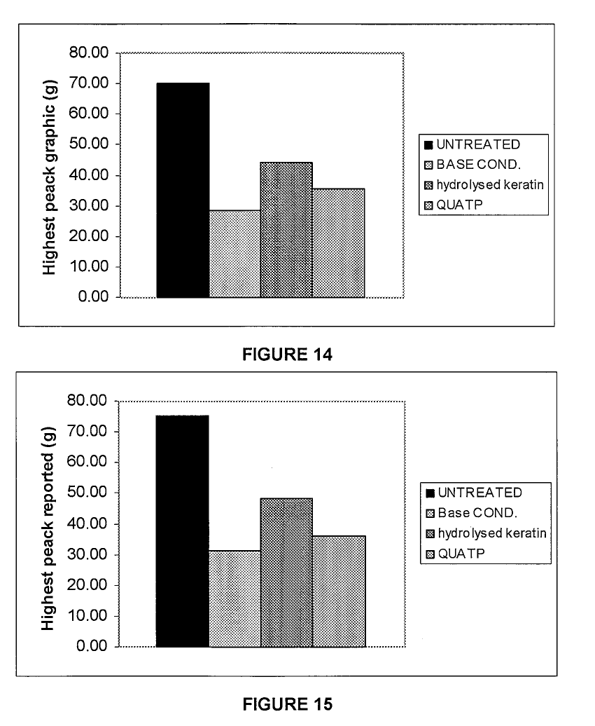

Tables 11-13 summarize the mean values found for two experiments completed to determine the combing parameters for the different hair samples. FIGS. 13-15 show the graphs for these results.

TABLE 11
Mean values for the measured combing force

| | | Base | Hydrolyzed | |
|---|---|---|---|---|
| | Untreated | Conditioner | Keratin | QUATP |
| Geometric | 42.08 | 17.44 | 28.90 | 24.27 |
| Average | | | | |
| [Combing Force/ | | | | |
| gF] | | | | |

As shown above, the results demonstrate the significant differences (t-student p<0.05) between the untreated and the treated hair samples on the combing force measured. All treatments lead to a decrease in the force required to comb the hairs which indicates an improvement in hair manageability. Evaluation of the different treatments show that the best results are due to the conditioner base treatment which decreases the combing force about 60% related to the untreated hair (significance difference, p<0.05) or about 30% less related to the rest of treatments (significance differences, p<0.05). No significant differences were found between the Keratec-Pep treatment and the QUATP treatment when considering mean combining force values.

TABLE 12
Mean values for the highest measured combing force

| | | Base | Hydrolyzed | |
|---|---|---|---|---|
| Comb no. | Untreated | Conditioner | Keratin | QUATP |
| Geometric | 70.10 | 28.33 | 44.09 | 35.30 |
| Average[Combing | | | | |
| Force/gF] | | | | |

TABLE 13
Mean values for the highest reported combing force

| | | Base | Hydrolyzed | |
|---|---|---|---|---|
| Comb no. | Untreated | Conditioner | Keratin | QUATP |
| Geometric | 75.49 | 31.38 | 48.36 | 35.30 |
| Average[Combing | | | | |
| Force/gF] | | | | |

Data for the highest peak (graphic and reported) also demonstrates that three treatments improve the hair manageability by reducing the force required to comb the hair comparing to the untreated values (significant differences, t-student p<0.05). Evaluation of the three different treatments shows that no significant differences are found between the conditioner base and QUATP conditioner treatments. Treatment with the conditioner base lead to slightly better results which gave a decrease about 58% in both combing force parameters related to the untreated hair values (t-student, p<0.05) and a decrease of approximately 35% when compared to the hydrolyzed keratin conditioner treatment values (t-student, p<0.05).

A panel test with 12 judges was used to evaluate the sensorial properties of the treated hair tresses. The tests were performed in a conditioned room (20° C. and 60% RH), where all four hair tresses (untreated and treated) were compared in pairs and the following questions were asked for each pair of samples:
1. Which hair tress is softer?
2. Which hair tress is smoother?
3. Which hair tress do you prefer?

All results were then subjected to statistics analysis: SPEARMAN'S RANK Correlation Coefficient was used to investigate the degree of agreement between judges and the Chi-Square Test was used to investigate if the volunteer's answers distributions differed from one to another.

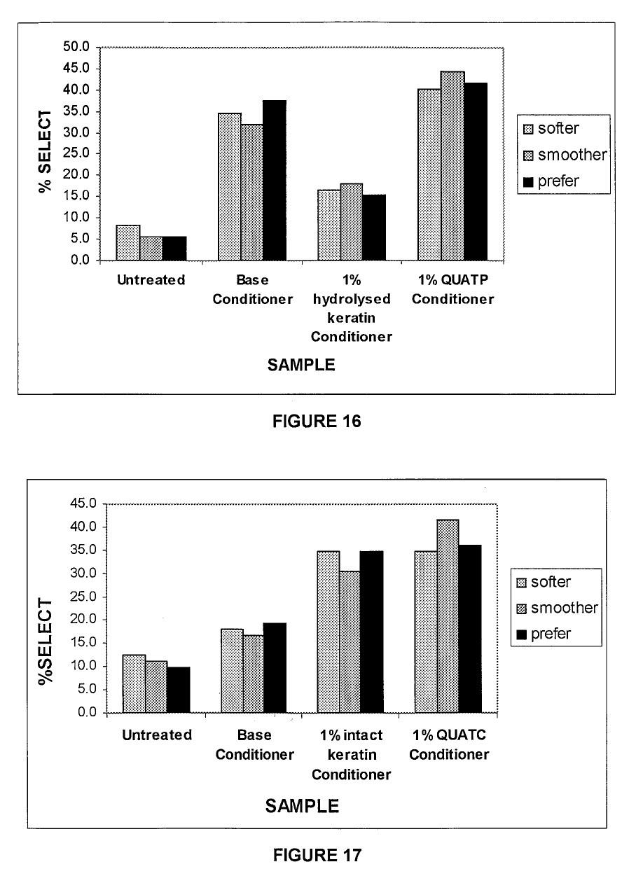

FIG. 16 shows the results for the selection percentage of the judges on the panel testing. The first statistical analysis indicated that in the three questions all the judges show a high degree of agreement (significance level p<0.05). Data demonstrates that there is a clear trend of the judges on selecting the QUATP conditioner and the conditioner base treated samples on the three different tests. Comparing these two samples, slightly better results are found for the QUATP conditioner treatment.

For test 1, results show that while 40% of the panel found the QUATP conditioner treated sample to be softer, 34% considered the conditioner base sample to be softer, 17% considered the hydrolyzed keratin conditioner sample to be softer and the final 8% thought that the untreated hair sample was the softest (significant differences (p<0.05) between untreated and QUATP conditioner treated samples).

In the second test it can be seen again that, while the highest percentage was for the QUATP conditioner treated sample, with the 44% of the judges choosing this as the smoother sample (significant differences, p<0.05, between QUATP conditioner and untreated and hydrolyzed keratin conditioner treated samples. No significant differences between QUATP and conditioner base treated samples), 32% opted for the conditioner base treated sample, 18% considered smoother the hydrolyzed keratin conditioner treated sample and 6% found the untreated sample the smoothest.

Finally, the same behavior was found in the last test were judges preferred the QUATP (with the 42%) and conditioner base treated (with the 38%) hair samples (significance differences p<0.05 related to the untreated and hydrolyzed keratin treated hair samples; No significant differences between them). While the lowest percentages were for the hydrolyzed keratin conditioner (15%) and the untreated (6%) hair samples.

### Trial Summary

The data confirms the conditioning effect on hair of the three different conditioners tested (QUATP conditioner, base conditioner and hydrolyzed keratin conditioner). This is demonstrated by a decreased combing force which reflects a healthier, more youthful hair surface and is associated with the consumer perception of better hair manageability.

Results also demonstrate that the inclusion of low molecular weight quaternised keratins doesn't show a significant improvement on hair conditioning compared with other conditioning agents. But comparing the two peptides treatments the QUATP peptide appears to perform better than the hydrolyzed keratin peptide.

### Example 9

### Influence of Intact Quaternised Keratin Derivative on Hair Physical Properties

The aim of this example was to evaluate the effect of intact quaternized keratin from wool on hair. The methods used in this Example were identical to Example 8 above except that the hydrolyzed quaternized keratin sample used in Example 8 (QuatP) was substituted with an intact quaternized keratin derivative in this example (termed QUATC and discussed above in Example 2).

Combing force results are shown below in Tables 14-16 averaging the two experiments completed.

TABLE 14
Mean values for the combing force measured

| | | Base | Intact | |
|---|---|---|---|---|
| Comb no. | Untreated | Conditioner | Keratin | QUATC |
| Geometric | 45.50 | 27.32 | 30.89 | 19.49 |
| Average | | | | |
| [Combing Force/ | | | | |
| gF] | | | | |

The measurements made demonstrate the significant differences (t-student p<0.05) between the untreated and the treated hair samples on the combing force measured. All treatments lead to a decrease in the force required to comb the hair which indicates an improvement in hair manageability. Evaluation of the different treatments show that the best results are due to the QUATC treatment which decreases the combing force about 55% related to the untreated hair (t-student, p<0.05) or about 30% less related to the rest of the treatments (t-student, p<0.05).

TABLE 15
Mean values for the highest peak measured

| | | Base | | |
|---|---|---|---|---|
| Comb no. | Untreated | Conditioner | Intact keratin | QUATC |
| Geometric | 76.00 | 40.17 | 45.10 | 29.05 |
| Average | | | | |
| [Combing Force/ | | | | |
| gF] | | | | |

TABLE 16
Means values for the highest peak reported

| | | Base | | |
|---|---|---|---|---|
| Comb no. | Untreated | Conditioner | Intact keratin | QUATC |
| Geometric | 81.91 | 45.18 | 52.55 | 34.56 |
| Average | | | | |
| [Combing Force/ | | | | |
| gF] | | | | |

Data for the highest peak (graphic and reported) also demonstrates that the three treatments improve the hair manageability by reducing the force required to comb the hair comparing to the untreated values (significant differences, t-student p<0.05). Evaluation of the three different treatments show that most significant results are found with the QUATC conditioner treatment which gave a decrease about 60% in both combing force parameters related to the untreated hair values (t-student, p<0.05) and a decrease of approximately 30% when compared to the other conditioner treatments (t-student, p<0.1).

FIG. 17 shows the results for the selection percentage of the judges on the panel testing. The first statistical analysis indicated that in the three questions all the judges show a high degree of agreement (significance level p<0.05). Data demonstrates that there is a clear trend of the judges on selecting the QUATC and intact keratin conditioners treated samples on the three different tests.

For test 1, results show that while the 70% of the answers chose the QUATC and intact keratin conditioners treated samples as being the softer, the 12% considered the untreated sample to be the softest sample (significant differences p<0.05 between untreated and protein treated samples) and 18% thought the conditioner base treated hair sample was the softest, although no significant differences were found between the different treatments.

In the second test it can be seen again that while the 70% of the judges chose the QUATC and intact keratin conditioners treated samples as being the smoother ones the 11% found smoother the untreated sample (significant differences p<0.05 between untreated and protein treated) and the 19% opted for the conditioner base treated sample. In this test a statistically significant difference can be found between treatments (significance level p<0.05) with the QUATC conditioner treated samples being selected as the most smooth by a total of the 42% of selection.

Finally, in the last test, judges were asked to choose which sample they preferred. The same behavior was found, with 71% of the judges choosing the QUATC and intact keratin conditioner treated samples, while 20% opted for the conditioner base treated sample leaving the 9% of the selection for the untreated sample (significant differences p<0.05 between untreated and protein treated, no significant differences between treatments).

### Trial Summary

This study demonstrates the conditioning effect of the intact quaternized keratin on hair. This is demonstrated by a decreased combing force which reflects a healthier, more youthful hair surface and is associated with the consumer perception of better hair manageability.

Aspects of the present invention have been described by way of example only and it should be appreciated that modifications and additions may be made thereto without departing from the scope thereof as defined in the appended claims.

What we claim is:

1. A soluble keratin derivative comprising a soluble keratin protein with at least one substituted chemical group at a point on the soluble keratin protein selected from the group consisting of: a lysine group; a terminal amine group; a hydroxyl amino acid group; and combinations thereof.

2. The soluble keratin derivative as claimed in claim 1 wherein the soluble keratin protein is intact.

3. The soluble keratin derivative as claimed in claim 1 wherein the soluble keratin protein is hydrolyzed.

4. The soluble keratin derivative as claimed in claim 1 wherein the substituted chemical group comprises a negatively charged group.

5. The soluble keratin derivative as claimed in claim 4 wherein the soluble keratin derivative comprises a soluble keratin succinylation derivative.

6. The soluble keratin derivative as claimed in claim 4 wherein the substituted chemical group comprises: 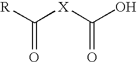
where R=the soluble keratin protein and X=an optionally substituted alkyl group.

7. The soluble keratin derivative as claimed in claim 6 wherein X=(CH₂)ₙ and n=2 to 6.

8. The soluble keratin derivative as claimed in claim 4 wherein the soluble keratin derivative comprises a soluble keratin fatty acid derivative.

9. The soluble keratin derivative as claimed in claim 4 wherein the substituted chemical group comprises: 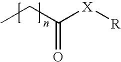
where R=the soluble keratin protein, X=NH or O, 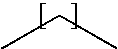
=a repeating fatty acid chain, and n=1 to 40.

10. The soluble keratin derivative as claimed in claim 9 wherein X=NH, 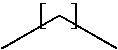
=(CH₂) and n=10 to 18.

11. The soluble keratin derivative as claimed in claim 1 wherein the substituted chemical group comprises a positively charged group.

12. The soluble keratin derivative as claimed in claim 11 wherein the soluble keratin derivative comprises a soluble keratin quaternisation derivative.

13. The soluble keratin derivative as claimed in claim 11 wherein the substituted chemical group comprises: 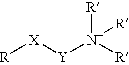
where R=the soluble keratin protein, X=NH or O, Y=an optionally substituted alkyl chain and R′=an alkyl chain.

14. The soluble keratin derivative as claimed in claim 13 wherein X=NH, Y=CH₂CH(OH)CH₂ and R′=CH₃.

15. The soluble keratin derivative as claimed in claim 1 wherein the soluble keratin protein is S-sulfonated.

16. The soluble keratin derivative as claimed in claim 1 wherein the soluble keratin protein comprises keratin intermediate filament protein fraction.

17. The soluble keratin derivative as claimed in claim 1 wherein the soluble keratin protein comprises keratin high sulfur protein fraction.

18. The soluble keratin derivative as claimed in claim 1 wherein the soluble keratin protein comprises keratin high glycine-tyrosine protein fraction.

19. A method of producing a soluble keratin derivative, the method comprising the step of completing a substitution reaction of a chemical group at a point on a soluble keratin protein selected from the group consisting of: a lysine group; a terminal amine group; a hydroxyl amino acid group; and combinations thereof.

20. The method as claimed in claim 19 wherein the soluble keratin protein is intact.

21. The method as claimed in claim 19 wherein the soluble keratin protein is hydrolyzed.

22. The method as claimed in claim 19 wherein the chemical group comprises a negatively charged group.

23. The method as claimed in claim 22 wherein the soluble keratin derivative comprises a soluble keratin succinylation derivative.

24. The method as claimed in claim 22 wherein the chemical group comprises: 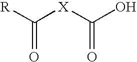
where R=the soluble keratin protein and X=an optionally substituted lower alkyl group.

25. The method as claimed in claim 24 wherein X=(CH₂)ₙ and n=2 to 6.

26. The method as claimed in claim 22 wherein the soluble keratin derivative comprises a soluble keratin fatty acid derivative.

27. The method as claimed in claim 22 wherein the chemical group comprises: 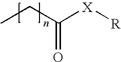
where R=the soluble keratin protein, X=NH or O, 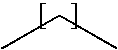
=a repeating fatty acid chain, and n=10 to 40.

28. The method as claimed in claim 27 wherein X=NH, 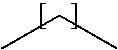
=(CH₂) and n=10 to 18.

29. The method as claimed in claim 19 wherein the chemical group comprises a positively charged group.

30. The method as claimed in claim 29 wherein the soluble keratin derivative comprises a soluble keratin quaternisation derivative.

31. The method as claimed in claim 29 wherein the chemical group comprises 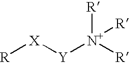
where R=the soluble keratin protein, X=NH or O, Y=an optionally substituted alkyl chain and R′=an alkyl chain.

32. The soluble keratin derivative as claimed in claim 31 wherein X=NH, Y=CH₂CH(OH)CH₂ and R′=CH₃.

33. The method as claimed in claim 19 wherein the soluble keratin protein is S-sulfonated.

34. The method as claimed in claim 19 wherein the soluble keratin protein comprises keratin intermediate filament protein.

35. The method as claimed in claim 19 wherein the soluble keratin protein comprises keratin high sulfur protein.

36. The method as claimed in claim 19 wherein the soluble keratin protein comprises keratin high glycine-tyrosine protein.

37. A surfactant product comprising a soluble keratin derivative, the soluble keratin derivative comprising a soluble keratin protein with at least one substituted chemical group at a point on the protein selected from the group consisting of: a lysine group; a terminal amine group; a hydroxyl amino acid group; and combinations thereof.

38. A personal care formulation comprising from about 0.001% to 50% by weight of a soluble keratin derivative.

39. The personal care formulation as claimed in claim 38 wherein the soluble keratin derivative comprises a soluble keratin protein with at least one substituted chemical group at a point selected from the group consisting of: a lysine group; a terminal amine group; a hydroxyl amino acid group; and combinations thereof.

40. The personal care formulation as claimed in claim 38 wherein the soluble keratin protein is intact.

41. The personal care formulation as claimed in claim 38 wherein the soluble keratin protein is hydrolyzed.

42. The personal care formulation as claimed in claim 38 wherein the substituted chemical group comprises a negatively charged group.

43. The personal care formulation as claimed in claim 42 wherein the soluble keratin derivative comprises a soluble keratin succinylation derivative.

44. The personal care formulation as claimed in claim 42 wherein the substituted chemical group comprises: 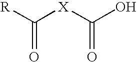
where R=the soluble keratin protein and X=an optionally substituted lower alkyl group.

45. The personal care formulation as claimed in claim 44 where wherein X=(CH₂)ₙ and n=2 to 6.

46. The personal care formulation as claimed in claim 42 wherein the soluble keratin derivative comprises a soluble keratin fatty acid derivative.

47. The personal care formulation as claimed in claim 42 wherein the substituted chemical group comprises: 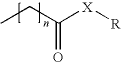
where R=the soluble keratin protein, X=NH or O, 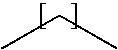
=a repeating fatty acid chain, and n=1 to 40.

48. The personal care formulation as claimed in claim 47 wherein X=NH 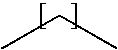
=(CH₂) and n=10 to 18.

49. The personal care formulation as claimed in claim 38 wherein the substituted chemical group comprises a positively charged group.

50. The personal care formulation as claimed in claim 49 wherein the soluble keratin derivative comprises a soluble keratin quaternisation derivative.

51. The personal care formulation as claimed in claim 49 wherein the substituted chemical group comprises: 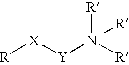
where R=the soluble keratin protein, X=NH or O, Y=an optionally substituted alkyl chain and R′=an alkyl chain.

52. The personal care formulation as claimed in claim 51 wherein X=NH, Y=CH₂CH(OH)CH₂ and R′=CH₃.

53. The personal care formulation as claimed in claim 38 wherein the soluble keratin protein derivative is S-sulfonated.

54. The personal care formulation as claimed in claim 38 wherein the soluble keratin protein comprises keratin intermediate filament protein fraction.

55. The personal care formulation as claimed in claim 38 wherein the soluble keratin protein comprises keratin high sulfur protein fraction.

56. The personal care formulation as claimed in claim 38 wherein the soluble keratin protein comprises keratin high glycine-tyrosine protein fraction.

57. An additive for a personal care formulation comprising a soluble keratin derivative, the soluble keratin protein derivative comprising a soluble keratin protein with at least one substituted chemical group at a point on the protein selected from the group consisting of: a lysine group; a terminal amine group; a hydroxyl amino acid group; and combinations thereof.

58. A method of treating hair or skin, the method comprising the step of applying a personal care formulation comprising from about 0.001% to 50% of a soluble keratin derivative.

59. A method of treating hair or skin by the step of applying a personal care formulation comprising an additive, the additive comprising a soluble keratin derivative, the soluble keratin derivative comprising a soluble keratin protein with at least one substituted chemical group at a point on the protein selected from the group consisting of: a lysine group; a terminal amine; a hydroxyl amino acid group; and combinations thereof.

60. A soluble keratin derivative mixture comprising:
a first soluble keratin protein fraction with at least one substituted chemical group at a point on the soluble keratin protein fraction selected from the group consisting of: a lysine group; a terminal amine group; a hydroxyl amino acid group; and combinations thereof; and
a second soluble keratin protein fraction with at least one substituted chemical group at a point on the soluble keratin protein fraction selected from the group consisting of: a lysine group; a terminal amine group; a hydroxyl amino acid group; and combinations thereof;
wherein the first soluble keratin protein fraction and the second soluble keratin protein fraction are each selected from the group consisting of intermediate filament protein, high sulfur protein and high glycine-tyrosine protein; and
wherein the first soluble keratin protein fraction is not the same as the second soluble keratin protein fraction.

61. A method of producing a soluble keratin derivative mixture, the method comprising the step of:
mixing a first soluble keratin protein fraction having at least one substituted chemical group at a point on the soluble keratin protein fraction selected from the group consisting of a lysine group, a terminal amine group, a hydroxyl amino acid group, and combinations thereof, with a second soluble keratin protein fraction having at least one substituted chemical group at a point on the soluble keratin protein fraction selected from the group consisting of a lysine group, a terminal amine group, a hydroxyl amino acid group, and combinations thereof;
wherein the first soluble keratin protein fraction and the second soluble keratin protein fraction are each selected from the group consisting of intermediate filament protein, high sulfur protein and high glycine-tyrosine protein; and
wherein the first soluble keratin protein fraction is not the same as the second soluble keratin protein fraction.

62. The soluble keratin derivative as claimed in claim 1 wherein the soluble keratin protein is partially oxidized.

63. The method as claimed in claim 19 wherein the soluble keratin protein is partially oxidized.

64. The personal care formulation as claimed in claim 38 wherein the soluble keratin protein derivative is partially oxidized.
